(19) United States
(12) Patent Application Publication
(10) Pub. No.: US 2023/0220487 A1
(43) Pub. Date: Jul. 13, 2023

(54) MOLECULAR MARKERS AND METHODS FOR SAMPLE ANALYSIS VIA MASS SPECTROMETRY

(71) Applicant: BOARD OF REGENTS, THE UNIVERSITY OF TEXAS SYSTEM, Austin, TX (US)

(72) Inventors: Livia S. EBERLIN, Austin, TX (US); Ruth KATZ, Houston, TX (US)

(73) Assignee: BOARD OF REGENTS, THE UNIVERSITY OF TEXAS SYSTEM, Austin, TX (US)

(21) Appl. No.: 18/001,425

(22) PCT Filed: Jun. 7, 2021
(86) PCT No.: PCT/US2021/036108

§ 371 Date: Dec. 9, 2022

Related U.S. Application Data
(60) Provisional application No. 63/038,593, filed on Jun. 12, 2020.

(51) Int. Cl. C12Q 1/6886; G01N 33/574

(52) U.S. Cl. CPC C12Q 1/6886; G01N 33/57492; C12Q 2600/16; C12Q 1/6841

(57) ABSTRACT

Methods for detecting cancer cells, or aggressive cancers, by measuring levels of cardiolipin molecules are provided. Methods of treating identified cancers are likewise provided.

### PRIORITY CLAIM

This application claims benefit of priority to U.S. Provisional Application Ser. No. 63/038,593, filed Jun. 12, 2020, the entire contents of which are hereby incorporated by reference.

### BACKGROUND

### 1. Field

The present disclosure relates generally to the field of molecular biology, organic chemistry and oncology. More particularly, it concerns lipid markers for disease lesions, such as cancer.

### 2. Description of Related Art

It is now firmly established through numerous studies that malignant cells that are derived from primary epithelial or mesenchymal tumors can extravasate into the peripheral blood stream to become circulating tumor cells or CTCs, which in turn become the “seeds” for implantation in distant organ sites (Pantel and Brakenhoff, 2004; Husemann et al., 2008; Rhim et al., 2012; Sanger et al., 2011; He et al., 2007; Pantel and Speicher, 2016). First described as a curiosity in the blood in 1869, by Thomas Ashworth based solely on cytological appearance, currently CTCs have assumed immense importance and attention in modern pathology and oncology, not only as a diagnostic and prognostic modality, but also as a novel vehicle to study the molecular pathogenesis and physiology of events leading to carcinogenesis (Hanahan and Weinberg, 2011). CTCs can be characterized further by molecular methods such as PCR or NGS on a single cell basis, as well as by FISH or IHC on intact circulating tumor cells, which has led to the very appropriate designation of blood as being a “liquid biopsy.” This terminology, which has now become integrated into the lexicon of oncology, refers to the fact that blood, unlike deep seated organs, such as lung, colon, and pancreas, is easy to access by a simple needle stick, which can be easily repeated on a serial basis, in order to detect or monitor the course of a patient's cancer.

Cell free tumor DNA (cftDNA). In addition, to CTCs patients with cancer can manifest cell free tumor DNA (cftDNA), liberated from the constant turnover of nuclear contents from CTCs, and their accompanying exosomes. Assessment of cell free DNA (cftDNA) in non-small cell lung cancer (NSCLC) by PCR has recently been FDA approved as a sensitive and reliable method to monitor the development of mutations such as the T790M, acquired by patients treated for EGFR mutations by first line tyrosine kinase inhibitors, necessitating a switch in targeted therapies. Unlike CTCs, cftDNA is easier to enrich from whole blood, but limited by the need to know the target mutations to confirm the origination from tumor cells (Scher et al., 2017). Recently next generation sequencing (NGS) using hybrid capture assays, have been used to detect multiple alterations simultaneously in a single test. Not only are known “hot spot” mutations detected, but the assay allows for detection of unknown alterations. In a study of 119 patients with advanced EGFR-TKI-naïve NSCLC and 15 EGFR-TKI-resistant patients, somatic cftDNA mutations by NGS, were detected in 82.8% of patients in the total cohort, of which 27.7% were suitable for approved targeted therapy drugs. Actionable genetic mutations included predominantly EGFR mutations, and the EGFR T790M mutation, as well as BRAF mutations, MET mutation and gene fusions for EML4-ALK and KIF5B-RET. In a smaller cohort, genomic alterations that could be targeted such as mTOR inhibitors, PARP inhibitors and CDK4/6 inhibitors were discovered. This study used a broad hybrid capture-based 508-gene panel NGS assay (Oseq-NT) in 10 ml of sample of peripheral blood (Hou et al., 2017).

Pathogenesis of CTCs. The pathogenesis of the origin of CTCs encompasses several of the hall marks of cancer (Hanahan and Weinberg, 2011). There are certain enabling characteristics in the evolution of malignancy that occur in cells, such as the emergence of unregulated proliferation secondary to acquisition of mutations leading to oncogenesis, or deletions or methylation of tumor suppressor genes that allow disturbance of the balance between pathways involved in the cell cycle of promoting growth, versus other pathways that promote suppression of growth or arrest of the cell cycle. A dominant pathway that drives tumor growth at the expense of cell cycle arrest or apoptosis may lead to replicative immortality. Additionally other mechanisms such as angiogenesis, a micro-environment of acute inflammation and macrophages, as well as the cells' ability to avoid immune surveillance via masking of checkpoint inhibitors and acquisition of altered metabolic pathways by increasing glucose uptake, will all add to the eventual acquisition of characteristics that allow invasion into the blood stream as a circulating blood cells or CTCs (Hanahan and Weinberg, 2011).

Clinical Applications of CTCs as Liquid biopsy. Clinically, enumeration of CTCs may be used for early diagnosis of a tumor, such as in the face of an indeterminate lung nodule in a patient at high risk for lung cancer, serial monitoring of a tumor's response to therapy where drop in CTCs would indicate response to therapy, single tumor cell sequencing to detect mutations, and transfer of CTCs into an animal model to develop patient derived xenographs or genetically engineered mouse models (GEMS) for culture and study of the pathophysiology of the tumor or responses to different therapies based on genomic findings (Pantel and Speicher, 2016; Ni et al., 2013)) Hence the study of CTCs allows the serial monitoring of tumor genotypes and may provide an insight into prognostic and predictive biomarkers for targeted therapy and clinical usage.

### SUMMARY

In accordance with the disclosure, there is provided a method of detecting a circulating tumor cell (CTC) in a fluid sample comprising (a) obtaining a cell-containing fluid sample from a subject, wherein said fluid sample is buffy coat portion of a blood sample; (b) performing an ambient ionization MS on the sample to obtain a profile for the sample; and (c) detecting the presence of a CTC based on the profile. The sample may be disposed on a surface and said method may further comprise marking one or more regions on said surface corresponding to a detected CTC.

In some aspects, the methods further comprise hybridizing the cells in said sample with labeled nucleic acid probes for (a) 3p22.1, 10q22.3, chromosome 10 centromeric (cep10) and (b) chromosome 3 centromeric (cep3) or 3q29 tel, and detecting CTCs based on pattern of hybridization to all four labeled nucleic acid probes to said selected cells. In some aspects, the label is a fluorescent label or a chromogenic label. In some aspects, the methods are further defined as a methods for detecting cancer in the subject. In some aspects, the CTCs are from cancer that gives rise to blood borne metastases, such as cancer of lung, head and neck, breast, thyroid colon, prostate, pancreas, esophagus, kidney, a gastro-intestinal tumor, a urogenital tumor, kidney, a melanomas, an endocrine tumor, a sarcoma, or circulating malignant cells derived from a leukemia or a lymphoma. In some aspects, the methods further comprise detecting hybridization of one or more additional labeled nucleic acid probes, such as a UroVysion DNA probe set, a LaVysion DNA probe set, a centromeric 7/7p12 Epidermal Growth Factor (EGFR) probe, cep7/7p22.1, cep17, and 9p21.3 probes, EGFR/cep and 10/cep10q probes, pTEN, cep10 and cep10q probes, an EML4-ALK probe set, a cytoplasmic probe such as microRNA probe such as miRNA21, a surface or cytoplasmic biomarker probe such as ER,PR, Her2neu, a pan-cytokeratin, or a CA19. In some aspects, the ambient ionization MS comprises DESI-MSI. In further aspects, the methods comprise performing 2D DESI-MSI. In still further aspects, the 2D DESI-MSI comprises a spatial resolution of 500 μm to 50 μm.

In some aspects, the methods further comprise obtaining a reference profile and detecting the presence of CTCs by comparing the profile from the sample to a reference profile. In further aspects, the reference profile is obtained from the same subject. In other aspects, the reference profile is obtained from a different subject. In some aspects, the profile comprises fatty acid and metabolite molecules and species with m/z of 215.033, 255.233, 283.264, and 303.233. In some aspects, the profile comprises ceramide species with m/z ratios of 572.48, 656.578, and 682.594. In other aspects, the profile comprises glycerophosphoethanolamine molecules and species with m/z 722.513, 750.546, and 766.542. In still other aspects, the profile comprises cardiolipin molecules and species with m/z 723.499, and 725.495. In yet other aspects, the profile comprises glycerophosphoserine molecules and species with m/z 788.545, and 810.529. In other aspects, the profile comprises glycerophosphoglycerol molecules and species with m/z 747.520, 773.534. In still other aspects, the profile comprises glycerophosphoinositol molecules and species with m/z 835.535, 857.520, 861.551, and 885.550.

In some aspects, the methods further comprise filtering the fluid sample. In some aspects, filtering comprises use of a vacuum apparatus and a membrane perforated with 7.5 μm pores. In some aspects, the buffy coat layer is separated from the blood by a Ficoll-Hypaque gradient. CTCs are normally distributed throughout the cellular fraction of the blood, and centrifugation over a gradient such as Ficoll-Hypaque allows the more buoyant cells, that is the cells with more cytoplasm, to band at the interface between the plasma and the Ficoll, due to the specific gravity of the mononuclear cells, lymphocytes, mononuclear cells such as histocytes, and CTCs. Hence, the so-called “buffy” coat is enriched for CTCs as the red blood cells and the neutrophils sink to the bottom of the tube, away from the buffy coat.

In some aspects, the buffy coat layer is further purified by CD45 bead-based purification to remove white blood cells. In other aspects, the buffy coat layer is further purified by CD3 bead-based purification to remove white blood cells. In some aspects, the buffy coat layer is separated from the blood by a Ficoll-Hypaque gradient, is further purified by CD45 bead-based purification to remove white blood cells and is further purified by CD3 bead-based purification to remove white blood cells. In some aspects, the methods further comprise collecting the sample from the subject.

In some aspects, the methods further comprise selecting CTCs by assessing nuclear area comprises determining pixel size for each CTC and applying a pre-determined threshold for exclusion, and or determining nuclear diameter and/or determining DAPI concentration and its standard deviation. In some aspects, detecting comprises assessing all abnormalities or gains only. In some aspects, the methods further comprise (c) administering at least a first anticancer therapy to a subject identified to have CTCs. In further aspects, the anticancer therapy comprises radiation, immunotherapy, toxin therapy, hormonal therapy, surgery or chemotherapy therapy. In still further aspects, the anticancer therapy is a combination therapy comprising more than one of radiation, immunotherapy, toxin therapy, hormonal therapy, surgery and chemotherapy therapy.

In another embodiment, the present disclosure provides methods of treating a subject comprising (a) selecting a patient determined to have CTCs in accordance with the methods set forth above; and (b) administering at least a first anticancer therapy to the subject.

In some aspects, the anticancer therapy comprises radiation, immunotherapy, surgery or chemotherapy therapy. In some aspects, the cancer is a one that gives rise to blood borne metastases, such as cancer of lung, head and neck, breast, colon, prostate, pancreas, esophagus, kidney, a gastro-intestinal tumor, a urogenital tumor, kidney, a melanomas, an endocrine tumor (thyroid, e.g., papillary thyroid cancer (PTC) adrenal gland cortex or medulla), a sarcoma, a leukemia or a lymphoma.

As used herein in the specification and claims, “a” or “an” may mean one or more. As used herein in the specification and claims, when used in conjunction with the word “comprising”, the words “a” or “an” may mean one or more than one. As used herein, in the specification and claim, “another” or “a further” may mean at least a second or more.

As used herein in the specification and claims, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating certain embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. CTC clusters in early breast cancer. Slides show entire repertoire of nucleated white blood cells, i.e., “no cell left behind” including clusters of CTCs and single CTC representing breast cancer cells. Note red box containing cluster of CTCs, from an early breast cancer patient.

FIGS. 2A-G. Diagram depicting methods for isolation and detection of CTCs. (FIG. 2A) Affinity-dependent detection devices usually employing magnetic particles, beads or posts coated with EpCAM such as a) CellSearch®, MACS or Mag-sweeper. (FIG. 2B) CTC chip Affinity-free detection devices relying on physical properties of CTCs such as larger size or deformability compared to other blood cells using (FIG. 2C) inertial focusing or (FIG. 2D) trapping of cells when passed through a Parasortix® filtration cassette or (FIG. 2E) enriched on a Ficoll-Hypaque gradient due to specific gravity and centrifugal forces and characterized based on genetic abnormalities by FISH such as the Sanmed™ Multiplex 4-color FISH test or (FIG. 2F) trapped on filtration membranes that only permit passage of white blood cells with a pore size smaller than CTCs such as Cell-Sieve, ISET™, Screen Cell™ Cyto and (FIG. 2G) total cell capture coupled with red blood cell lysis and immunocytochemistry or FISH together with high resolution imaging for cell morphology for detection of CTCs, such as the Epic or Tethis SBS platforms.

FIGS. 3A-F. FICTION, combined immunohistochemistry with genetic abnormalities depicted by FISH probe centromeric 10 and 10q22.3. (FIG. 3A) CD45−/CK+ showing gain of 10q22.3. (FIG. 3B) CD45+/CK− gain of 10Q22.3. (FIG. 3C) cancer stem cells CD45−/ALDH1+, monosomy 10 in CD45− cell, and gain of 10q in ALDH1+ cell. (FIG. 3D) CD45+E/CK−, monosomy 10 and gain of 10q22.3. (FIG. 3E) CK+/CD45− gain of 10q. (FIG. 3F) SNAIL 1+/CD45− with monsomy 10 and gain of 10q22.3 showing EMT differentiation.

FIG. 4. Patient 29, 61-year old female, stage IIIA adenocarcinoma, deceased.

FIGS. 5A-D. (FIG. 5A) Patient with stage IV adenocarcinoma of lung presenting with metastasis to the eye. FISH at Cep10/10q22.3, combined with ALDH staining on resected tumor, depicted a single ALDH1+ stained stem cell (white arrow) showing deletion of 10q22.3 (FICTION on tissue section, ALDH1 immunofluorescent stain with DAPI counterstain; ×400 objective). (FIGS. 5B-C) CTC from the baseline blood of the same patient before surgery, expressing ALDH1 and monosomy Cep10/10q22.3, and also co-expressing CK (red, FIG. 5Da) and ALDH1 (green, FIG. 5Db).

FIG. 6. Circulating cytogenetically abnormal cells (Y axis) over time (X axis), stage 1B NSCLC, patient alive 3 years later, EMT peak after surgery, drop back to baseline.

FIG. 7. CD45− (yellow arrow) and CD45+ cells (blue arrow) with gains of 10q22.3(RED signals) PBMNC's from patient with small cell carcinoma of lung.

FIGS. 8A-C. Filter preps of malignant cells isolated from blood and pleural fluid, and then subjected to multiplex FISH, showing aneuploidy.

FIGS. 9A-G. Methods of enrichment using both positive selection (anti-epithelial or EPCAM ferrofluids as in CellSearch) with silicone microposts or chips coated with anti-EPCAM ferrofluids (FIG. 9A, FIG. 9D), and negative selection via beads coated with anti-CD45 for leukocyte depletion (FIG. 9B). (FIG. 9C) Filtration relying on physical properties, where cells that are smaller than 8 μm, such as hematolymphoid cells, are depleted, leaving larger CTCs to remain on top of the filter; the examples are ISET and CellSieve. (FIG. 9E) PBMNCs containing the CTC fraction are enriched via gradient centrifugation from which cytospin slides can be prepared for IHC, and FISH studies for DNA and RNA. (FIG. 9F) Electric field diaphoresis or DEP arrays to separate large cells via a non-uniform electric field using physical properties to trap cells of interest in DEP cages (Menarini-Silicon Biosystems). (FIG. 9G) A microfluidic device relying on a single spiral microchannel to separate CTCs from other blood components.

FIGS. 10A-F. Microcavitary array system.

FIG. 11. Binding of the EpCAM Monoclonal Antibody (mAb) ferromagnetic conjugate to EpCAM on CTC cell membrane.

FIGS. 12A-B. Microfluidic chip device. System isolates CTCs based on physical size of cells into orifices on device. Confirmation of CTCs is performed in situ by combination of CK, Hoechst dye and CD45

FIG. 13. The principle of combining FISH with IHC or FICTION to provide a window into discovering immunophenotypic diversity and chromosomal alterations. Ficoll-purified cells are first reacted with fluorescent-tagged antibodies of interest, scanned on an automated fluorescent microscope, and the locations of positive and negative cells are captured. The slide is then reacted with appropriate DNA probes by FISH, rescanned, and cells of interest are matched and analyzed. comprising CK8,18,19/CD45 in conjunction with a battery of 12 specific DNA FISH probes targeting chromosome regions that had previously been shown to be aberrantly altered in NSCLC. Inventors were able to demonstrate four different immunofluorescent-phenotypic subpopulations of genetically abnormal cells as follows: (1) negative for both epithelial and lymphoid markers or “double-negative” (DN) cells, most likely representing cells that had undergone EMT or were stem cells; (2) cells that co-expressed CK and CD45 (CK+/CD45+); (3) cells expressing only CK (CK+/CD45−), and (4) cells expressing only CD45 but not CKs (CD45+/CK−).

FIG. 14. Combined FISH and Immunohistochemistry FICTION.

FIG. 15. CTCs, polysomies, pancreatic cancer (top panel). Diff Quik binucleated cells post chemotx with taxanes (bottom panel).

FIGS. 16A-E. CTCs lung cancer post resection of tumor showing GLUT-1 positivity.

FIGS. 17A-B. identification of somatic mutations and copy number alterations in individual CTCs. (FIG. 17A) Visualization of genome coverages and aligned reads around the EGFR gene in 3 CTCs and 1 WBC using an integrative genome viewer. A 15-bp deletion in exon 19 of the EGFR gene was detected in all CTCs but not the WBC. (FIG. 17B) CNA patterns of a WBC, 4 CTCs from a lung cancer patient, and 1 CTC each from patients with different types of cancer (prostate, breast, colon, gastric). The copy numbers (blue and red dots) are plotted along the genome at a bin size of 500 kb. The ordinate co-ordinate represents copy numbers ranging from 0 to 6 (a copy number of more than 6 copies is set to 6).

FIGS. 18A-D. CTCs from triple negative breast cancer patient with brain metastasis.

FIGS. 19A-D. Ex vivo cultured CTCs from breast cancer patients metastatic to brain in mice (FIG. 19A, FIG. 19C) and from patient's brain metastasis (FIG. 19B, FIG. 19D).

### DESCRIPTION OF ILLUSTRATIVE EMBODIMENTS

### I. THE PRESENT EMBODIMENTS

Studies detailed herein provide new methodologies for detecting cancer cells and, in particular, oncocytic tumor cells. In particular new lipid marker of the cancer cells were identified. In the studies herein mass spectroscopy, DESI-MS, was used to image and chemically characterize the lipid composition of thyroid tumors. The analysis revealed a novel molecular signature in oncocytic tumors characterized by an abnormally high abundance and chemical diversity of CL species. DESI-MS imaging and IHC experiments confirmed that the spatial distribution of these molecular ions overlapped with regions of accumulation of mitochondria-rich oncocytic cells. Moreover, fluorescence imaging confirmed that the oncocytic tumors investigated presented high accumulation of mitochondria when compared to non-oncocytic and normal thyroid tissue.

Using high-mass accuracy, high-mass resolution, and tandem MS experiments, 101 CL species directly from oncocytic thyroid tissues were identified. Amongst the CL species identified, 54 doubly charged molecular ions composed of CL bound to PC or DG were seen at high relative abundances in oncocytic tumors when compared to non-oncocytic or normal thyroid tissues. Likewise, 17 different ox-CL were identified in oncocytic tumors. Oxidization of other abundant polyunsaturated phospholipids were not observed in the studies, which indicates that this phenomenon is primarily occurring for CL in oncocytic tumors themselves.

Thus, the studies herein provide new means for identification of cancer cells, such as oncocytic thyroid tumor cells, or cells having mitochondrial dysregulation by detecting abnormal expression and composition of CL and other lipids. In particular, MS detection of CL and CL oxidation products can be used to generate a profile indicating the presence of lesions in a patient. The presence of these profiles can then be used to guide patient therapy. For example, in the case of a patient identified to have an oncocytic tumor, a more aggressive therapy regime can be used to address the cancer. Thus, the methodologies and markers provided herein should provide a new avenue for accurate diagnosis and treatment for cancers, such as thyroid cancers.

### II. ASSAY METHODOLOGIES

In some aspects, the present disclosure provides methods of determining the presence of a tumor by identifying specific patterns of lipids such as cardiolipins. These patterns may be determined by measuring the presence of specific lipid ions using mass spectroscopy. Some non-limiting examples of ionizations methods include chemical ionization, atmospheric-pressure chemical ionization, electron ionization, fast atom bombardment, electrospray ionization, and matrix-assisted laser desorption/ionization. Additional ionization methods include inductively coupled plasma sources, photoionization, glow discharge, field desorption, thermospray, desorption/ionization on silicon, direct analysis in real time, secondary ion mass spectroscopy, spark ionization, and thermal ionization.

In particular, the present methods may be applied to an ambient ionization source or method for obtaining the mass spectral data such as extraction ambient ionization source. Extraction ambient ionization sources are methods with a solid or liquid extraction processes dynamically followed by ionization. Some non-limiting examples of extraction ambient ionization sources include air flow-assisted desorption electrospray ionization (AFADESI), direct analysis in real time (DART), desorption electrospray ionization (DESI), desorption ionization by charge exchange (DICE), electrode-assisted desorption electrospray ionization (EADESI), electrospray laser desorption ionization (ELDI), electrostatic spray ionization (ESTASI), Jet desorption electrospray ionization (JeDI), laser assisted desorption electrospray ionization (LADESI), laser desorption electrospray ionization (LDESI), matrix-assisted laser desorption electrospray ionization (MALDESI), nanospray desorption electrospray ionization (nano-DESI), or transmission mode desorption electrospray ionization (TM-DESI). In some embodiments, the ionization source used in the methods described herein is desorption electrospray ionization.

DESI is an ionization technique used to prepare a mass spectra of organic molecules or biomolecules. The ionization technique is an ambient ionization technique which uses atmospheric pressure in the open air and under ambient conditions. DESI is an ionization technique which combines two other ionization techniques: electrospray ionization as well as desorption ionization. Ionization is affected by directing electrically charged droplets at the surface that is millimeters away from the electrospray source. The electrospray mist is then pneumatically directed at the sample. Resultant droplets are desorbed and collected by the inlet into the mass spectrometer. These resultant droplets contain additional analytes which have been desorbed and ionized from the surface. These analytes travel through the air at atmospheric pressure into the mass spectrometer for determination of mass and charge. One of the hallmarks of DESI is the ability to achieve ambient ionization without substantial sample preparation.

As with many mass spectroscopy methods, ionization efficiency can be optimized by modifying the spray conditions such as the solvent sprayed, the pH, the gas flow rates, the applied voltage, and other aspects which affect ionization of the sprayed solution. In particular, the present methods contemplate the use of a solvent or solution which is compatible with human issue. Some non-limiting examples of solvent which may be used as the ionization solvent include water, methanol, acetonitrile, dimethylformamide, an acid, or a mixture thereof. In some embodiments, the method contemplates a mixture of acetonitrile and dimethylformamide The amounts of acetonitrile and dimethylformamide may be varied to enhance the extraction of the analytes from the sample as well as increase the ionization and volatility of the sample. In some embodiments, the composition contains from about 5:1 (v/v) dimethylformamideacetonitrile to about 1:5 (v/v) dimethyl-iformamideacetonitrile such as 1:1 (v/v) dimethylformamideacetonitrile.

Additionally, two useful parameters are the impact angle of the spray and the distance from the spray tip to the surface. Generally, the electrospray tip is placed from about 0.1-25 mm from the surface especially from 1-10 mm. In some embodiments, a placement from about 3-8 mm is useful for a wide range of different application such as those described herein. Additionally, varying the angle of the tip to the surface (known as the incident angle or α) may be used to optimize the ionization efficacy. In some embodiments, the incident angle may be from about 0° to about 90°. In some aspects, a poorly ionizing analytes such as a biomolecule will have a larger incident angle while better ionizing analytes such as low molecular weight biomolecules and organic compounds have smaller incident angle. Without wishing to be bound by any theory, it is believed that the differences in the incident angle results from the two different ionization mechanisms for each type of molecule. The poorly ionizing biomacromolecules may be desorbed by the droplet where multiple charges in the droplet may be transferred to the biomacromolecule. On the other hand, low molecular weight molecules may undergo charge transfer as either a proton or an electron. This charge transfer may be from a solvent ion to an analyte on the surface, from a gas phase solvent ion to an analyte on the surface, or from a gas phase solvent ion to a gas phase analyte molecule.

Additionally, the collection efficiency or the amount of desorbed analyte collected by the collector can be optimized by varying the collection distance from the inlet of the mass spectrometer and the surface as well as varying the collection angle (β). In general, the collection distance is relatively short from about 0 mm to about 5 mm. In some cases, the collection distance may be from about 0 mm to about 2 mm. Additionally, the collection angle (β) is also relatively small from about 1° to about 30° such as from 5° to 10°.

Each of these components may be individually adjusted to obtain an sufficient ionization and collection efficiencies. Within the DESI source, the sample may be placed on a 3D moving stage which allows precise and individual control over the ionization distance, the collection distance, the incident angle, and the collection angle.

Finally, the mass spectrometer may use a variety of different mass analyzers. Some non-limiting examples of different mass analyzers include time-of-flight, quadrupole mass filter, ion trap such as a 3D quadrupole ion trap, cylindrical ion trap, linear quadrupole ion trap, or an orbitrap, or a fourier transform ion cyclotron resonance device.

### III. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Methods

Sample Preparation. The sample treatment protocol employs 10 mL of blood collected from patients with advanced lung cancer, or healthy patients with no history of lung cancer (control group). The blood sample is split into 2 vials of 5 mL each for more efficient extraction. A density gradient is performed with various forms of density gradient mediums (e.g., Histopaque or Lymphoprep). These vials are then centrifuged to separate out each component of the blood sample (plasma, buffy coat, red blood cells, and platelets, etc.). The sample is washed with saline or water. The buffy coat is removed from both vials via pipette and cytospun onto one slide.

An alternative protocol could include a “no cell left behind” method where, following lysis of RBCs from whole blood, residual cells are deposited on slides with minimal manipulation, ensuring preservation of cell membranes that might occur with a cytocentrifuge.

In this protocol, all of the steps are relevant for subsequent successful detection of the CTC profile by MS. This includes the volume of sample, collection of the buffy coat layer, the density gradient, centrifugation, type of wash and the cytospin preparation. After this sample preparation step, the samples are shipped on dry ice to the University of Texas at Austin. This step is also relevant and preserves the cell quality for MS analysis.

Control Sample Preparation. Cultured cells from the A549 lung adenocarcinoma cell line (ATCC, Manassas, Va.) were spiked at concentrations of 1% and 10% into peripheral blood mononuclear cell (PBMC) obtained from control participants' blood samples and enriched by Ficoll-Hypaque density centrifugation.

CTC Quantitation. PBMC/A549 samples were deposited on microscope slides using a Shandon Cytospin 3 (Thermo Fisher Scientific). FISH was performed using the custom four-color probe set described in the methods. CTC were quantified using the same definition of CTC as described in the methods (i.e., polysomy of two or more genetic regions). To be counted, slides had to have at least 85% of interphase nuclei with optimum hybridization in the target area. Two experienced FISH technologists who were blinded to participants' cohort assignments analyzed each slide using Bioview software (Billerica, Mass.) optimized to display only mononuclear cells with a diameter greater than that of the average lymphocyte within the cytospin. Five hundred oval to round, intact, non-overlapping cells with excellent hybridization signals were analyzed. Loss and/or gain for CEP10 and subtelomeric 3q29 (1964F) probes were used as internal control probes. For each specimen,CTC number was recorded per 500 PBMC counted.

Total number of genetic abnormalities (including single gains, single deletions and CTC) were recorded per 500 PBMC counted. In undiluted A549 cells, 99.8% of cells analyzed were CTC (Table 2). In healthy control PBMC, no CTC were identified. The 1% spiked cell solution yielded 0.8% CTC frequency; the 10% solution yielded.

Ficoll-Hypaque Procedure for Peripheral Blood. Store blood at room temperature for processing. Add 5 ml of Histpaque (Sigma-Aldrich) to 15 ml Centrifuge tube, gently layer 3-4 ml of whole blood on top (save at least 200 μl of whole blood). Centrifuge at 1800 rpm for 20 minutes (300 G). Pipette off the Buffy coat into a clean 4 ml centrifuge tube. Fill tube to 15 ml with 1× PBS (1× PBS with 2 mM EDTA) and centrifuge at 1200 rpm for 10 minutes. Pipette off the supernatant, gently vortex the cell pellet and resuspend in 1× PBS (1× PBS with 2 mM EDTA) bringing volume to 1-2 ml. Perform a cell count on suspension and the saved whole blood. Adjust the dilution of the suspension so that there are no more than 40,000 cells/100 μl. Prepare one Diff Quick cytospin with 100 μl of above dilution to determine cellularity of the cells and make sure there are no overlapping cells. Make cytospins using a Cytocentrifuge at 750 rpm for 3 minutes. Depending on the concentration of cells make at least 12-24 cytospins including one Pap and one Diff Quick. Sprayfix immediately (SAFETEX Cytology Spray Fixative, Andwin Scientific, Woodland Hill, Calif.) Store in the appropriate study box in −20° C. freezer.

Fluorescence In situ Hybridization Protocol. Fix slides with Carnoy's fixative before pretreatment. Pre-heat the solutions at the recommended temperatures before starting the pre-treatment of slides. Immerse slides in 2×SSC for 2 minutes at 74° C. Then, immerse slides in Protease solution (25 mg protease II in 50 ml of 1× PBS at pH 2.00) at 37° C.×4 minutes. Wash slides in 1× PBS for 5 minutes. Fix slides in 1% Formaldehyde (0.5 ml formaldehyde in 50 ml of 1× PBS)×5 minutes. Wash slides in 1× PBS for 5 minutes. Dehydrate slides by immersing in 70%, 85% and 100% ethanol solutions for a minute in each respectively.

Hybridization. Add 10 μl of probe solution, using 1.5 μl from each color per slide (pretreated & dehydrated FISH slides). Cover with 12 mm round cover slip and apply rubber cement. Keep slides in Hybrid machine, program it for melting temp 73° C. for 2 minutes and then 37° C. for 18-20 hours to overnight. Alternatively, one can use Hybrid Moat for melting temperature at 73° C.×2 minutes and then place slides in a humidified box and place this box in an incubator set at 37° C.

Post-hybridization wash and staining. Pre heat 50-60 ml of post-wash solution at 74° C. in a water bath. Remove slides from the Hybrid machine/incubator and gently remove the rubber cement and cover slip. Immerse slides in 0.4×SSC/0.3% NP-40 for 2 minutes at 74° C. Then, immerse slides in 2×SSC/0.1% NP-40 at Room temperature for 1 minute. Drain and dry the slides for at least 30 minutes. Counter stain with 10 μl of DAPI II, cover slip, add immersion oil and visualize under fluorescent microscope using Triple, Green, Aqua and Gold filters.

Cell Sieve method of filtration. Blood samples were collected and diluted with equal amount of CellSieve prefixation buffer. Tubes were mixed by inverting three times and incubated for 15 minutes at room temperature.

Assembling of CellSieve microfilter and setting up of Filtration System is performed according to manufacturer's recommendations (Gabriel et al., 2016). Prefixed sample is then transferred to the inlet syringe in the filtration system Sample is passed through the filter device at the rate of 5 ml/minute by a “CellSieve Pull” program When the whole sample is passed through the filter device the system is turned off (Maheswaran and Haber, 2010).

Filter device is washed three times by adding 1× PBS into the filter holder opening. Filter holder is then disassembled according to the manufacturer's instructions and filter is removed with the help of a tweezer and placed on a microscope slide. At this step filters can be post fixed for immunostaining or FISH protocols.

### Example 2

### Results

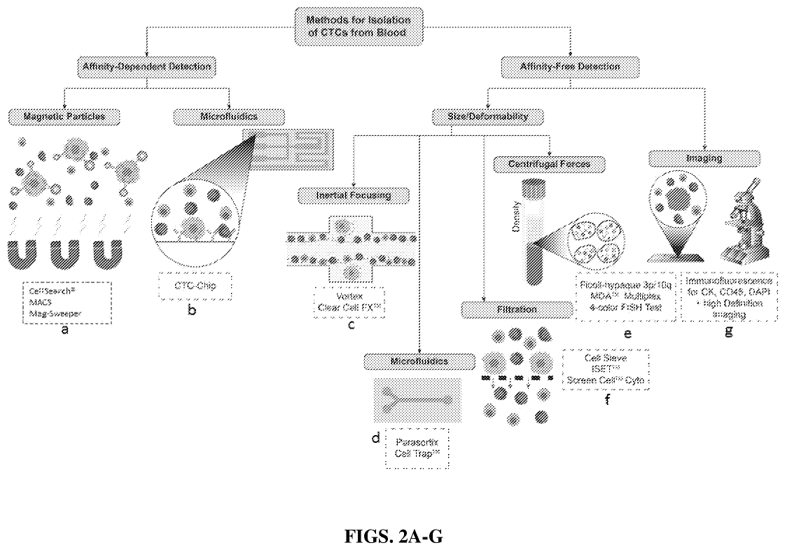

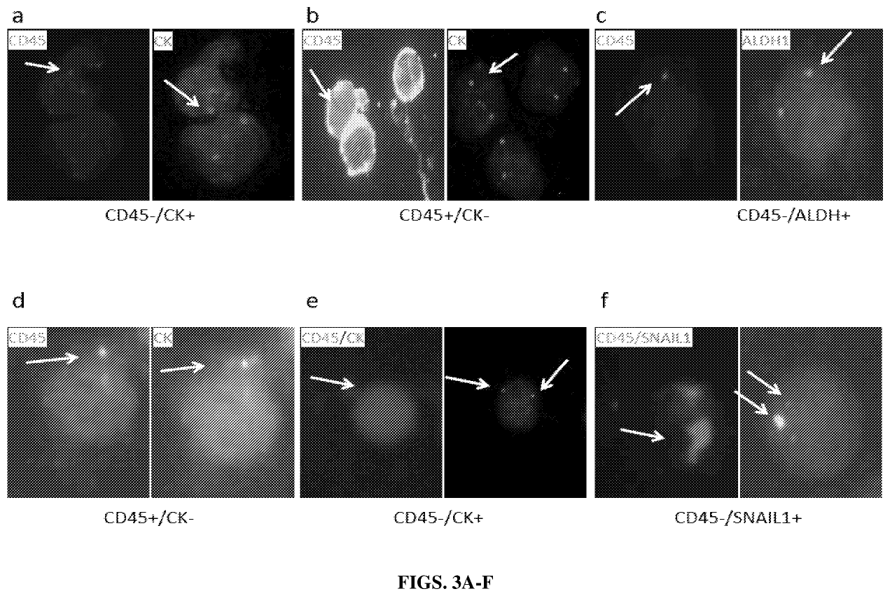

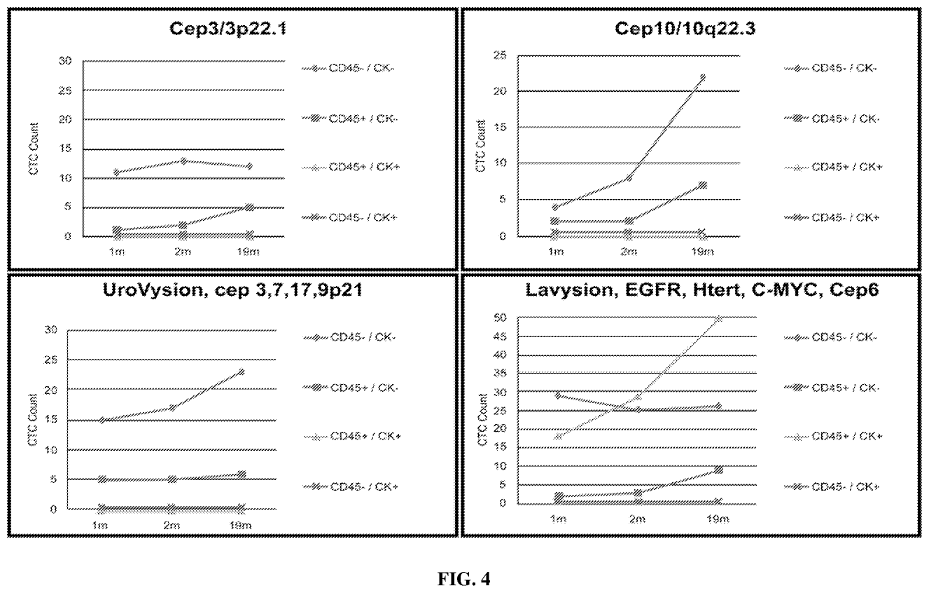

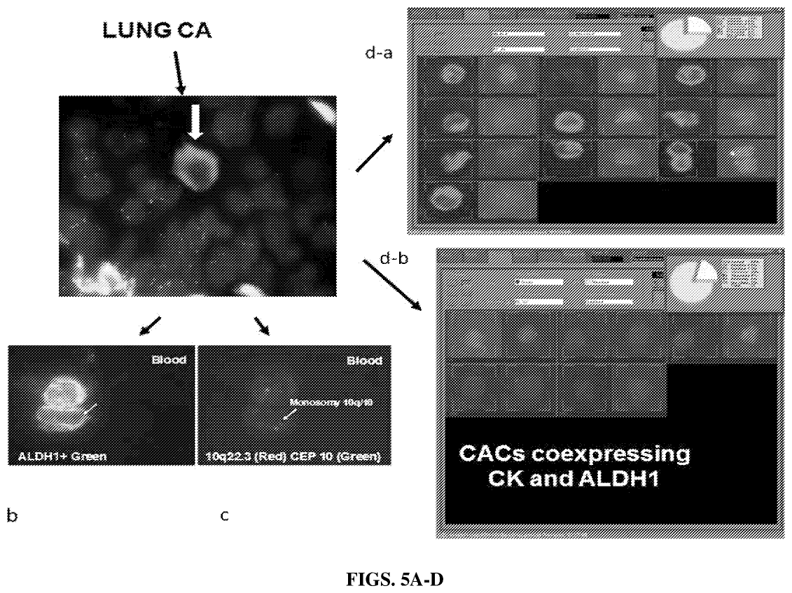

Composition of blood cells and numbers of CTCs. CTCs are extremely rare events in the peripheral blood stream, and depending on the method used to evaluate the numbers of CTCs such as the Cell Search Method, may range between 2 CTCs in patients with early stage disease breast cancer, to >5 CTCs per ml of blood in patients with high stage disease (Zhang et al., 2013). This is compared to lymphocytes and neutrophils that usually comprise up to several hundred million cells per ml (see FIG. 2), and RBCs which comprise one billion cells per ml, while there are several hundred thousand million platelets per ml of blood. In a previous study, using an antigen independent method to enumerate cytogenetically abnormal cells (CACs) by FISH, patients with NSCLC had significantly higher numbers of CACs than did controls (Katz et al., 2010). Mean numbers of CACs ranged from 7.23±1.32/μl for deletions of surfactant protein A gene relative to centromeric 10 (10q22.3/CEP10) to 45.52±7.49/μl for deletions of EIF1B, eukaryotic translation initiation factor relative to centromeric 3 (3p22.1/CEP3). Numbers of CACs with deletions of 3p22.1, 10q22.3, and 9p21.3 increased significantly from early to advanced stage of disease. These numbers of cytogenetically abnormal cells are far higher than those reported for the Cell Search Instrument and can be accounted for by the different method of enumeration, in which all cytogenetically abnormal cells were counted, irrespective of immunophenotype. These included cancer stem cells, malignant epithelial cells that had undergone epithelial mesenchymal transition (EMT cells) as well as CTCs of epithelial origin (CK-positive cells). Initially in early stage lung cancer, the vast majority of abnormal cells are CK-negative and CD45-negative, and may express EMT or stem cell markers (FIGS. 3A-F), hence the discrepancy between the FISH method and the Cell Search, where in the latter method, usually only a very low number of CTCs that are EpCAM-positive/CK-positive and CD45-negative are reported in a low percentage of NSCLC patients⁹). Using the FISH/FICTION method in patients with NSCLC who survived only a short time following a baseline blood sample, cytogenetically abnormal cells (CACs) were predominantly CK-positive/CD45-positive or CD45−/CK− (FIG. 4). In another patient who presented with adenocarcinoma metastatic to the eye(initially thought to be metastatic melanoma) baseline blood showed around 8 to 9 percent CACs which were CK+/ALDH1+ and around 10% of CACs that were CD45+/SNAIL1+ cells. These cells displayed an “intermediate phenotype” that is a phenotype co-expressing both epithelial or mesenchymal or stem cell markers such as CK , SNAIL1 andALDH1 (FIG. 5).

It has been demonstrated that in addition to single CTCs, cells may break off from the primary tumor in clusters and travel in clusters. CTC clusters may consist of 2 to more than 50 cells, when they are referred to as tumor micro emboli (TMC). There are reports that these TMCs may be associated with more malignant or aggressive properties based on findings that they may be protected from destruction by being enveloped by cells comprising those derived from the tumor microenvironment such as platelets, lymphocytes, neutrophils and macrophages (Krebs et al., 2014; Krebs et al., 2012; Hou et al., 2012).

Half-life of CTCs and tumor dormancy. Previous studies have focused on patients with breast cancer whose tumor cells can lie dormant for many years, in the bone marrow, before patients relapse with recurrent breast cancer. Based on the rapid disappearance of or drop in CTCs from the peripheral blood following resection, some authors have estimated that the half-life of breast cancer cells is from 2 to 4 and half hours.¹² The concept of “tumor self-seeding” by CTCs was first proposed by Massague and coworkers, who showed that CTCs mediated by tumor-derived cytokines 11-6 and 11-8, and in a breast cancer model, the chemokine CXCL1, a leukocyte-attractant cytokine, can colonize their tumors of origin. It was proposed that local recurrence could be produced by recirculation and self-seeding of more aggressive CTCs that can accelerate tumor growth, angiogenesis and stromal recruitment.^{12,13,14,15,16,17,18}

Concept of lineage plasticity. An advantage of sampling blood sequentially for CTCs, is that CTCs may manifest heterogeneity or mutations or deletions of key genes that drive unrestricted cellular proliferation compared to the main tumor mass and may manifest stem cell properties as well as EMT properties that are resistant to therapy. In order for cancer cells to metastasize, it is postulated that they need to adopt an EMT phenotype. In order to circulate, the cells derived from epithelial tumors undergo lineage plasticity or change their genotypic and phenotypic characteristics. In a previous label-free study of peripheral blood mononuclear cells (PBMNs), the inventors demonstrated by fluorescence in situ hybridization (FISH) that there were much higher numbers of circulating cytogenetically abnormal cells (CACs) in patients with lung cancer, and breast cancer across all stages, than had previously been reported by other methodologies (Katz et al., 2010; Zhang et al., 2013). These included the Cell Search assay, which isolates CTCs based on a bead-based antibody capture system for epithelial adhesion molecules (EpCAM), and where CTCs are defined as EpCAM captured cells that express cytokeratins but are negative for CD45 (Zhang et al., 2013). Because the FISH assay identified far higher numbers of CTCs in the blood stream of patients in both early and advanced NSCLC compared to the low numbers of CTCs reported by the EpCAM immunoantibody-cell capture methods (Katz et al., 2010; Maheswaran and Haber, 2010). The inventors postulated that the CTCs they had identified based solely on aneuploidy represented diverse cohorts of pluripotential CTCs. Thus CTCs may present as genetically abnormal cells with loss of epithelial, mesenchymal and hematopoietic markers or as cells that have undergone (EMT) with loss of epithelial markers but expression of mesenchymal markers, such as SNAIL, TWIST, ZEB or vimentin, or as cells that express only epithelial markers, such as EpCAM or cytokeratins with or without co-expression of CD45.

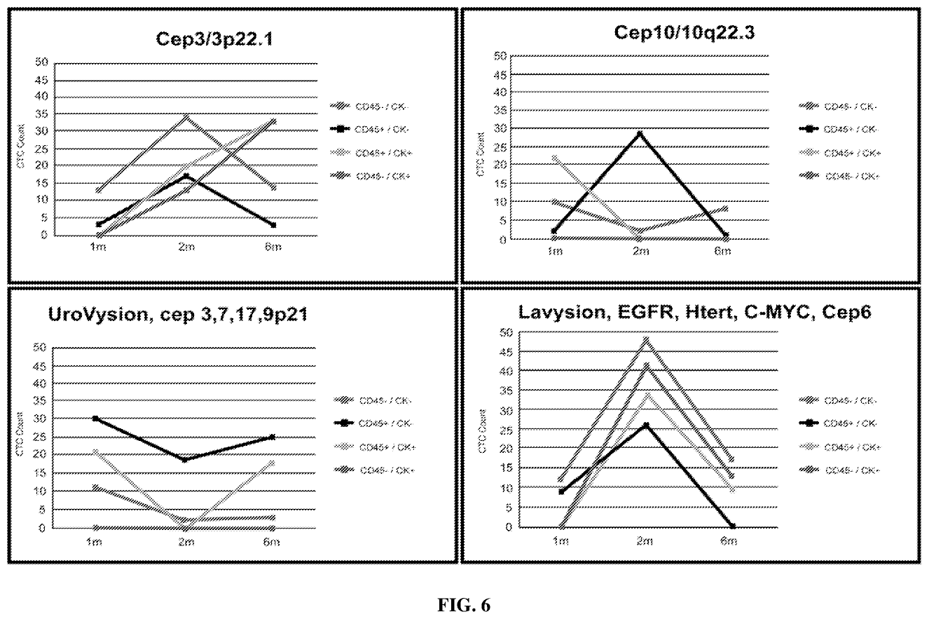

The inventors developed a lineage-labeling system which when combined with specific FISH genetic markers, was be able to detect and track both epithelial and mesenchymal CTCs over different time points (Katz et al., 2020). This system allowed them to correlate the kinetics of different cell phenotypes over time in patients both before surgery and in those who survived for moderately long periods of time after surgery (FIGS. 4 and 6).

Using a method known as FICTION (FIG. 4), the inventors evaluated PBMNCs with a dual-immunofluorescence (IFL) cocktail comprising CK8,18,19/CD45 in conjunction with a battery of 12 specific DNA FISH probes, previously shown to be aberrantly expressed in NSCLC (Katz et al., 2010). By using FICTION, they were able to demonstrate four different IFL-phenotypic subpopulations of genetically abnormal cells as follows:
(1) negative for both epithelial and lymphoid markers or “double-negative” cells (DN) most likely representing cells that had undergone stem cell or EMT
(2) cells that co-expressed cytokeratin and CD45 (CK+, CD45+)
(3) cells expressing only cytokeratin (CK+/CD45−)
(4) cells expressing only CD45 but not cytokeratins (CD45+, CK−)

An example of these different subtypes of circulating tumor cells expressing both immunohistochemical markers as well as abnormal gains or losses of different DNA probes is demonstrated (FIGS. 3A-F, FIG. 4) and is a typical pattern of progression. Here, the inventors show a patient who presented with advanced stage NSCL cancer who showed increasing CTCs over time, including increasing numbers of EMT/Stem cells showing abnormalities of 3p22.1 and 10q22.3, as well as increasing numbers of CK-positive cells showing aneuploidy for EGFR, CMYC, HTERT and Cep6. In contrast, patients who had long-lasting remissions following resection of NSCLC, showed in general a distinct peak in EMT and DN CTCs, up to 6 weeks post resection of tumor, and then over time all CTCs fell back to baseline levels (FIG. 6). The inventors believe that this latter phenomenon can be ascribed to extravasation of CTCs from sequestered sites following the resection of the primary lung cancer, whereafter CTCs disappear from the circulation over time.

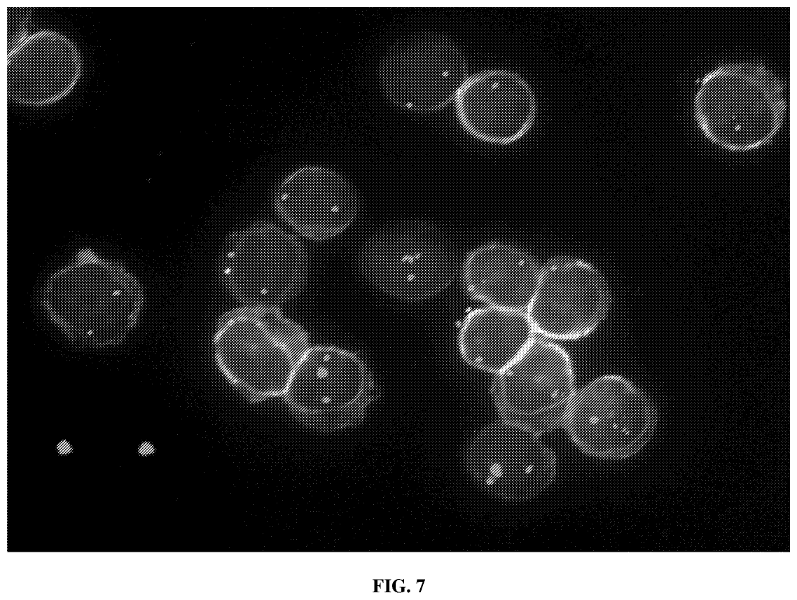

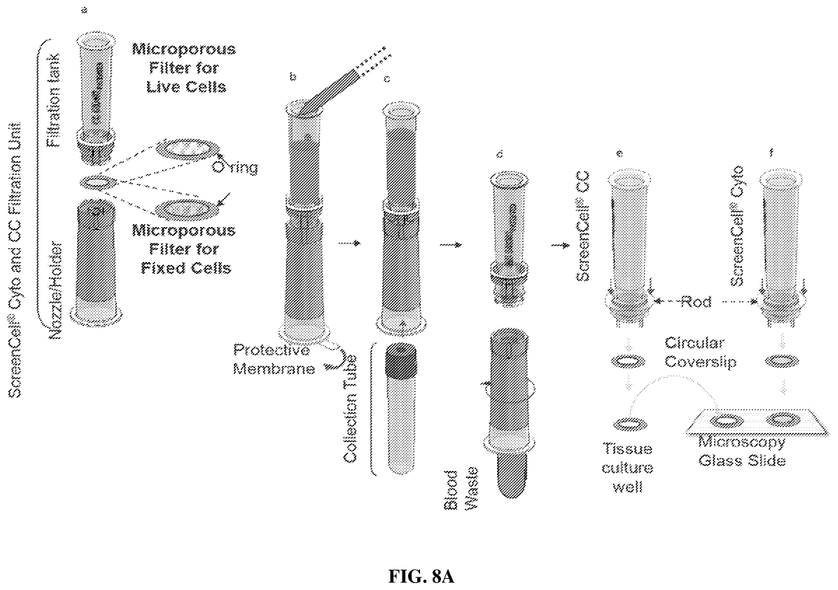

Remarkable was the consistent finding of cytogenetically abnormal cells with expression of CD45, or loss of expression of CD45 (FIG. 7) or co-expression of CD45 and other phenotypes such as SNAIL1, CK or ALDH1 (FIGS. 3A-F, FIG. 5). The phenomenon of “CTCs” co-expressing cytokeratin and CD45 has previously been noted by others (Zhang et al., 2013) who chose to exclude the presence of these cells from their enumeration based on their uncertain identity. However, based on the inventors' findings that these cells express genetic abnormalities identical to the other cell lineages, the inventors believe that cells that are CK+/CD45+ represent a significant “intermediate” phenotype of CTCs (FIG. 7).

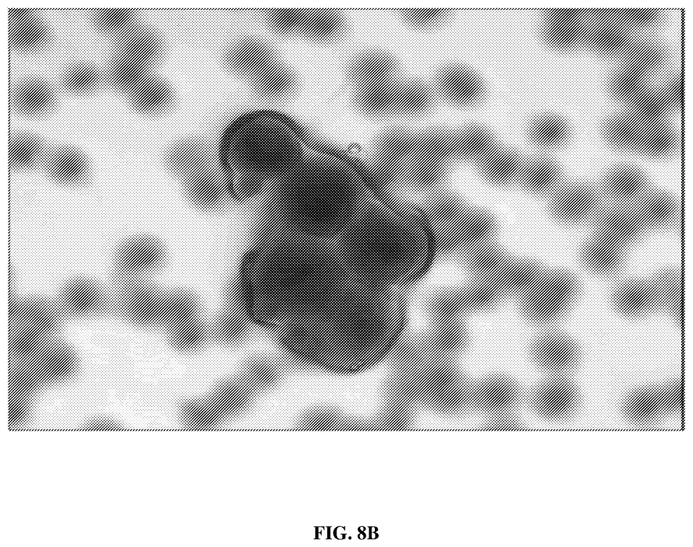

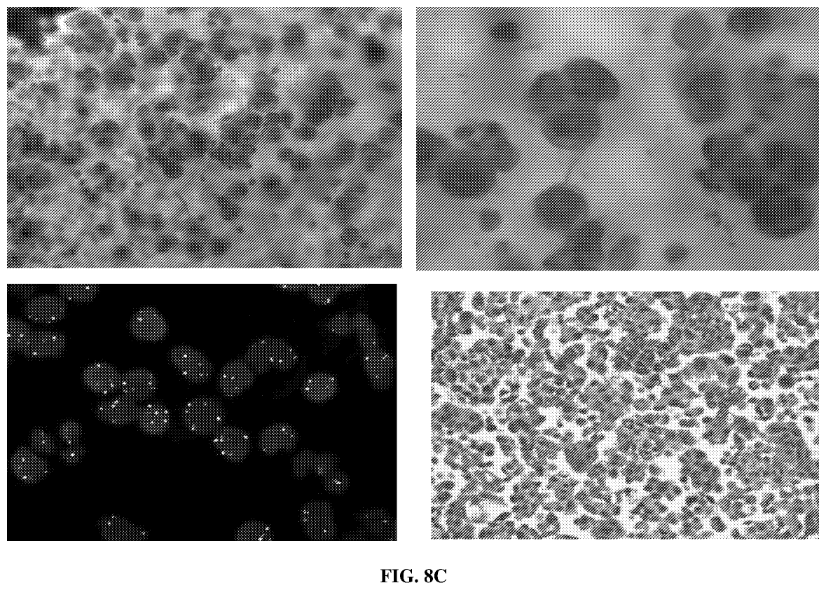

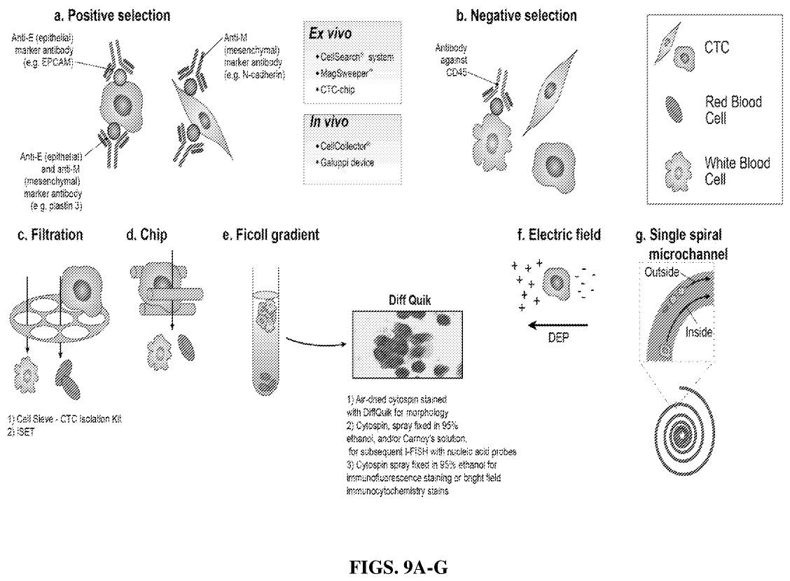

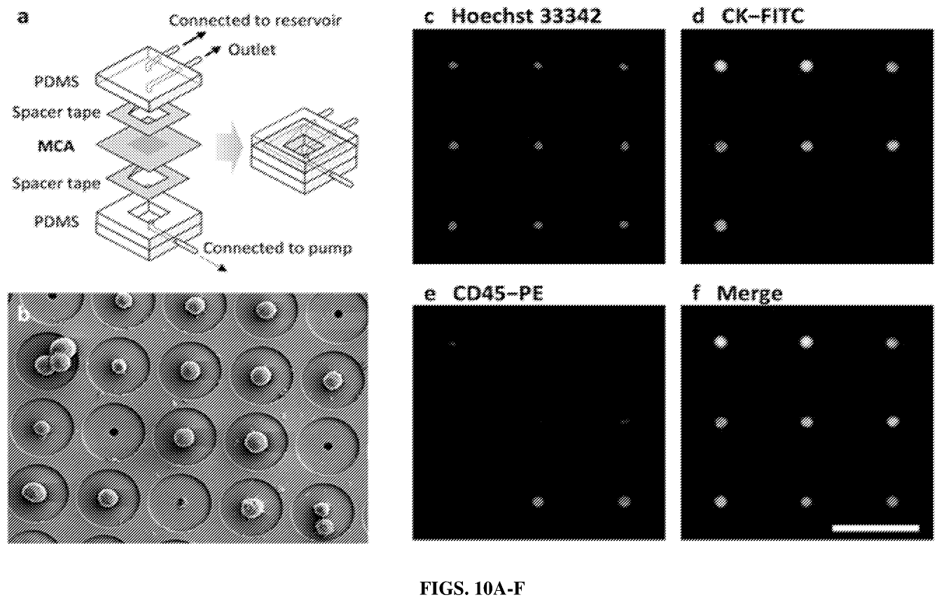

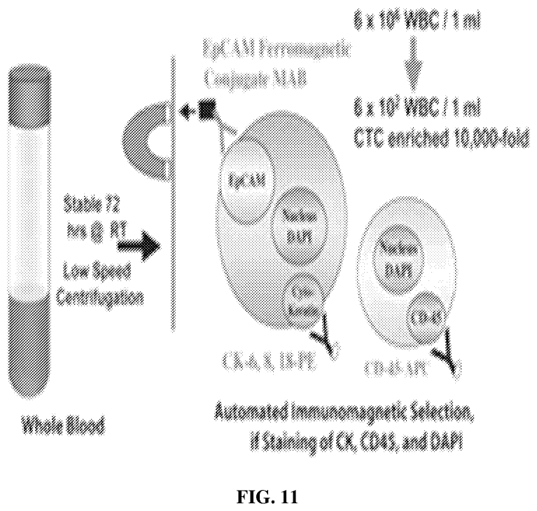

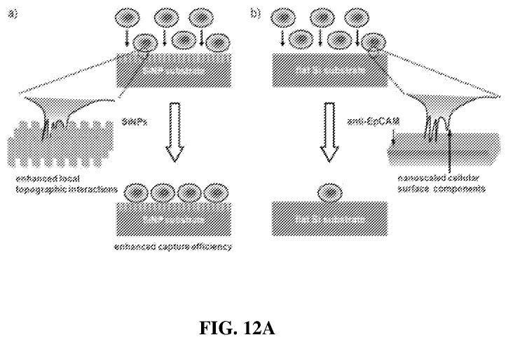

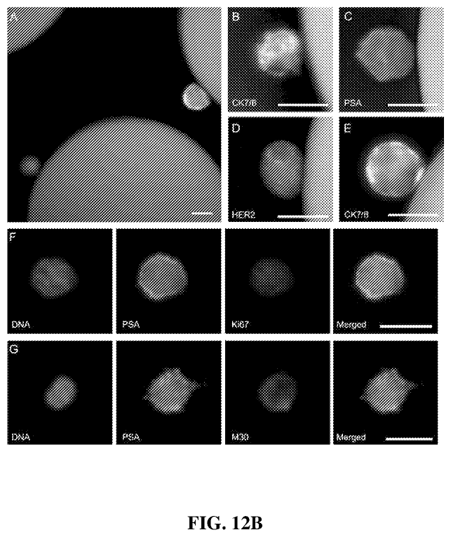

Types of platforms used for detection of CTCs. Currently there are numerous commercial endeavors to develop a method or a device that can maximize tumor cell yield in a consistent way even in the earliest stages of a malignant tumor. Some devices utilize enrichment methods that range from methods that can enrich the numbers of CTCs located in the buffy coat by several log units by a simple gradient centrifugation. Other methods require filtration of 10-15 ml of diluted blood through a membrane with small pores, just greater than the diameter of a lymphocyte, aided by a syringe pump, like CellSieve™ microfilters and filtration system from Creativ MicroTech, Inc.¹⁶ Many of the filtered large cells may be CTCs which can be verified by staining the filter membrane in situ for cytokeratin or performing FISH on the cell membrane to demonstrate aneuploidy or a gene of interest, such as a translocation of ALK-EML in the case of a non-small cell lung cancer (FIG. 8). However, complicating the identification of CTCs by these filtration methods, may be the concomitant recovery of many large mononuclear white blood cell or monocytes which may be difficult to identify from bone fide CTCs based solely on morphology. The different platforms that have been developed to isolate CTCs can be divided into antigen-dependent methods and antigen-independent or label-free methods and include microfluidic devices (FIGS. 3A-F, FIG. 9D, FIG. 9F, FIG. 9G, FIG. 11, FIG. 12) These latter methods may also rely on the physical properties of the CTCs, which are larger and less deformable or “stiffer” than the surrounding white blood cells such as DEP arrays. Methods employing physical properties of CTCs that allow CTC separation without labeling include (a) density gradient centrifugation such as Ficoll Hypaque gradient separation, often used to enrich the blood specimen by buoyant density of CTCs prior to selecting cells from the buffy coat for FISH or immunohistochemistry (FIG. 9E) (b) a microfluidic device combining multi-orifice flow fractionation (MOFF) and a dielectrophoretic (DEP) cell separation technique and (c) a dielectrophoretic field-flow fractionation (DEP-FFF) device allowing isolation of viable CTC by different response to DEP due to difference in size and membrane properties (FIG. 12)¹⁷. Another method employing physical properties is a microcavitary array system to trap cells based on the size of the orifice, and subsequently confirmed by immunohistochemistry for CK,CD45, and Hoechst 33342 (FIG. 10).

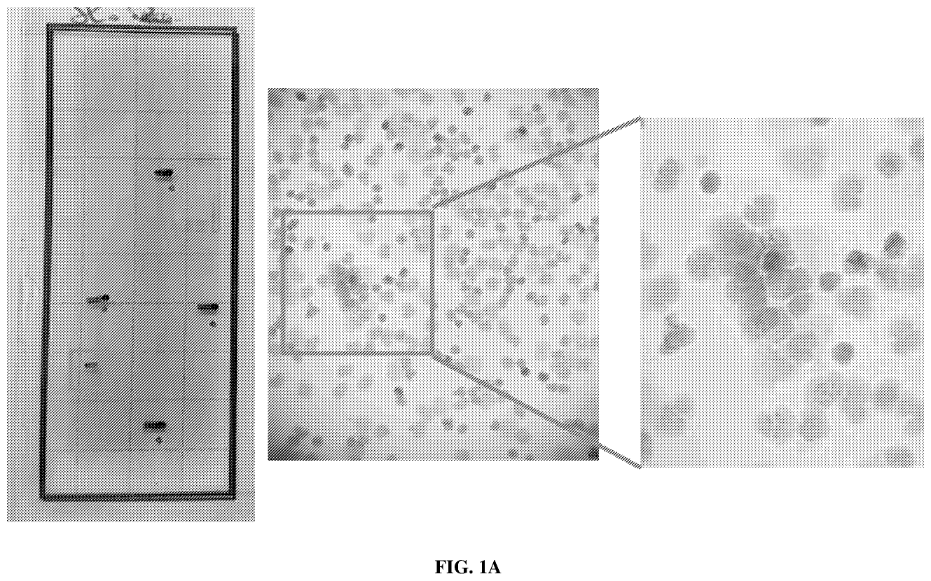

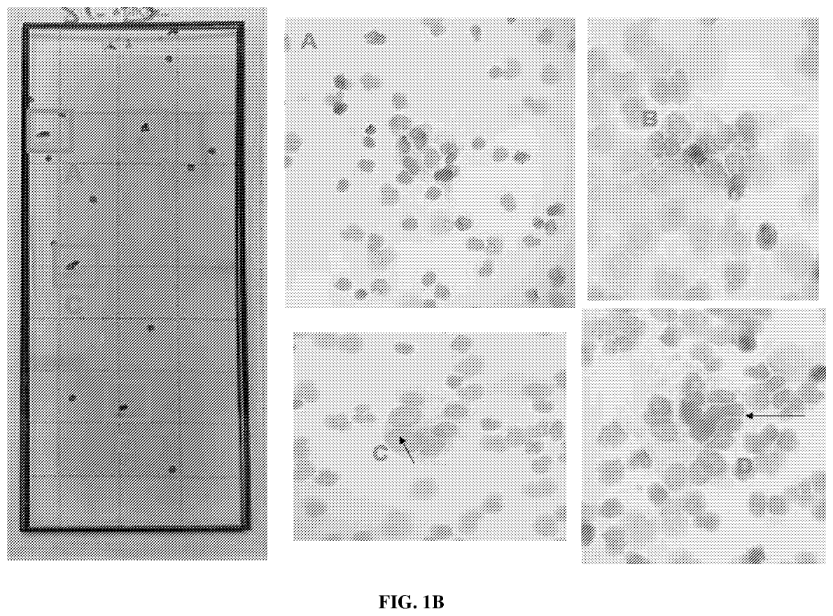

Other label-free methods for identifying CTCs include imaging methods that promote total tumor cell capture by minimal sample manipulation examining all nucleated cells in the blood by immunofluorescence (IF) for different antigens and tumor markers such as CK, ER, AR, Her2, or CD45 or FISH for aneuploidy and high-definition imaging for cell morphology (such as the EPICTM test or Tethis SBS Platform) (FIGS. 1A-B). See Ilona Krol et al., Detection of Clustered Circulating Tumor Cells in Early Breast Cancer, BJC 2021, in press; Scher H I, Graf R P, Schreiber N A, et al., Phenotypic Heterogeneity of Circulating Tumor Cells Informs Clinical Decisions between AR Signaling Inhibitors and Taxanes in Metastatic Prostate Cancer. Cancer Res 2017; 77:5687-98.

Antigen dependent devices or methods Immunomagenetic capture Isolation of CTCs is still not standardized; however, the Cell Search® System (Menarini-Silicon Biosystems, San Diego, Calif.) was acquired from Janssen Diagnostics in 2016. Outside of the United States, the test is made available by Janssen Diagnostics, a Veridex partner. is the only test platform for isolating CTCs that is FDA approved for the detection of breast, prostate and colorectal CTCs in patients with metastatic disease. The isolation principle is based on Ferro-beads coupled with an antibody to EpCAM, which is able to capture circulating tumor cells of epithelial origin. Captured cells are confirmed as CTCs by staining positive for a cocktail of cytokeratin's (CK8, 18 and 19) and staining negative for CD45, a lymphoid marker, to prove the epithelial nature of the cell. Thereafter, the cell, which should be greater than 5 μm in diameter, is stained with DAPI (4,6-diaminidino-2-phenylidole) for demonstrating the nucleus of the cell. The specimen requirement is 7.5 ml of whole blood, collected in special Cellsave® tubes and if the total number of cells meeting the afore-mentioned criteria is greater than 5, then the specimen is considered positive (FIG. 11) demonstrates principles of anti-EPCAM coated magnetic beads used in the Cell Search device. The prognostic significance of this test has been proven in studies of breast cancer where the median time of survival of patients with positive samples of CTCs≥5 or more, is half the median survival of patients with CTCs less than 5 (Miller et al., 2010). This device, although used extensively has a number of drawbacks regarding sensitivity for recovering and capturing the whole dynamic range of plasticity that CTCs may display as they access the circulation from the primary tumor because the Cell Search instrument in its conventional set up, is incapable of detecting cells that have lost or down regulated their EpCAM expression. It is known that numerous malignant cells on exiting the primary tumor will have undergone epithelial-mesenchymal transition or EMT resulting in loss or down regulation of surface epithelial antigens like EpCAM or cytokeratins. Thus, in many studies of solid tumors, zero or only 1-2 cells circulating tumor cells can be recovered by the Cell Search instrument in its conventional set-up (Zhang et al., 2013; Reithdorf et al., 2010; de Bono et al., 2007). Cancer cells that have undergone EMT are the source of metastases and in many tumors such as lung cancer, pancreatic cancer and breast cancer that metastasize to brain, the predominant component of CTCs are of the EMT type (Zhang et al., 2013).

A disadvantage of using the manufacturer's recommended tubes is a loss of EpCAM retrievable cells by a factor of 10, compared to other methods such as MAINTRAC™ using only anti-EpCAM in a preservative free collection tube such as EDTA and no enrichment procedures. It has also been shown that destruction of cell morphology by the preservative contributes to the poor cell recovery in the Cell Search system as compared to MAINTRAC (Pachmann et al., 2011).

Microfluidic devices based on 78,000 micro posts paced at very narrow intervals, forcing cells to move along narrow channels and enhancing their opportunities for contact with posts coated with EpCAM have been developed (FIG. 2D, FIG. 9G, FIG. 12) (Zhang et al., 2013), but may not be suitable for routine clinical use based on prolonged time required for specimen flow through and labor intensive confirmation that these represent epithelial cells. However, this CTC-chip enabled a higher yield of capture than the CellSearch instrument, (median 50 CTCs per milliliter), while the on-chip lysis feature, allowed for extraction of DNA and RNA and molecular analysis (Zhang et al., 2013).

Bead based subtraction-enrichment strategies Immunomagnetic positive enrichment methods commonly rely on epithelial antigens such as EpCAM for capture and tumor cell antigens such as cytokeratin for detection. The numbers of leftover leucocytes from these positive selection approaches were low ensuring a relatively pure population of CTCs for downstream analyses. However, EpCAM-negative CTCs, comprising mostly CTCs of non-epithelial origin or CTCs undergoing epithelial-mesenchymal transition, have been identified in peripheral blood in a few studies (Mikolajczyk et al., 2011; Grover et al., 2014).

To circumvent the false-negative CTC detection due to the absence of specific epithelial or tumor specific antigens in the surface of CTCs, negative selection approaches have been developed for unbiased CTC enrichment (Gabriel et al., 2016; Liu et al., 2011). Immunomagnetic selection with a cocktail of antibodies against hematopoietic antigens are utilized to enrich for CTCs by removing contaminating leukocytes. A few negative systems are commercially available. The RosetteSep (STEMCELL Technologies) depleted hematopoietic cells and platelets directly from whole blood with an antibody cocktail recognizing CD2, CD14, CD16, CD19, CD45, CD61, CD66b and Glycophorin A. Unwanted cells can also be removed by AutoMACS Separator (Miltenyi Biotech) with desired antibodies (FIG. 2B).

One major disadvantage of negative selection approaches is the lower CTC purity as compared to the positive selection approaches. It is difficult to identify a common marker for diverse lineages of hematopoietic cells. A cocktail of more antibodies could increase the specificity by depleting more leukocytes but will reduce the sensitivity for CTC detection owing to higher CTC binding, such as CTCs that are CK+/CD45+. Nevertheless, negative selection approaches show promise for identifying more CTCs for downstream analyses. Both epithelial and mesenchymal cancer cells could be enriched from patient samples as well as blood samples spiked with cancer cell lines (Lapin et al., 2016). CTCs enriched by negative selection were suitable for downstream analyses such as high-resolution genomic profiling. An advantage of this approach is that the assay can be performed in blood collected in fixative.

Antigen Independent Platforms. Enrichment Free Platforms or “No Cell Left Behind”. This method is based on capturing and analyzing all nucleated cells in a blood sample so that it is bias free and fully representative. Such a test has been developed at Epic Science® and is able to identify (a) traditional CTCs (CK+, CD45−, abnormal morphology), (b) CK−/CD45− CTCs (abnormal morphology, may be cancer stem cells or cells undergoing EMT), (c) apopototic CTCs (abnormal cells described in a and b, but with nuclear fragmentation, and the ratio of apoptotic CTCs to traditional CTCs may be indicative of response to therapy), and (d) CTC clusters (2 or more individual cells bound together, may be CK+ or negative, and may be associated with more aggressive metastatic potential than single cells). Following lysing of red blood cells (RBCs), nucleated cells are deposited on slides and each cell, following a cocktail of cytokeratin, CD45 and DAPI staining is analyzed for digital pathology features such as nuclear and cytoplasmic area, nuclear and cytoplasmic convex areas and major and minor axes. Additionally, other nuclear and cytoplasmic features, including but not limited to circularity, solidity, entropy, N:C ratio and nucleoli are analyzed (Scher et al., 2017). Machine learning clustering algorithms can then quantify different CTC subtypes into different categories to achieve an index of CTC heterogeneity. Cells of interest are then presented and confirmed by a trained operator as to whether they represent CTCs.

Other antibodies to proteins of interest, in addition to CK, can be analyzed and combined, such as a cocktail of antibodies against cytokeratin and an androgen receptor (AR), with co-expressing cells analyzed, and co-ordinates captured for future single cell analysis. In this case the coverslip can be lifted and the cell of interest is lifted off the slide for whole genome amplification (WGA) and NGS. In one particular study, the level of CTC heterogeneity was measured against survival times when patients were treated with taxanes for prostate cancer and demonstrated that the degree of CTC heterogeneity was a significant factor and correlated with overall survival (Scher et al., 2017).

An advantage of the Epic Science test is that the prepared slides can be stored in a biorepository at −80° C. and are able to be archived for future testing.

Antigen independent methods. Fluorescence in situ hybridization or FISH. In another antigen independent method, several thousand purified cells from the buffy coat of 10 ml of fresh blood, collected in an EDTA tube, can be subjected to interphase fluorescence in situ hybridization (I-FISH) with multiple DNA probes labeled with different fluorescent tags⁹ in order to identify nuclei containing gains or polysomies and deletions of different targeted genes (FIG. 9E).

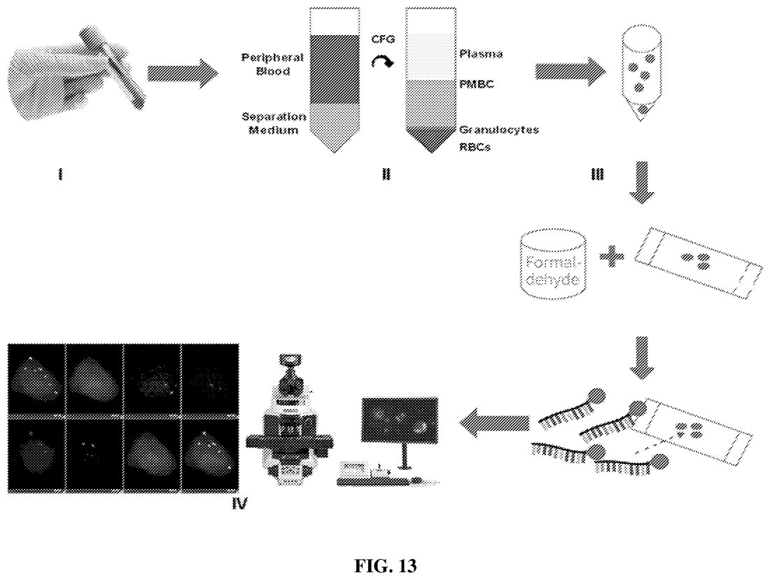

In order maximize the yield of CTCs; it is essential to first remove the background contaminating WBCs, which comprise the vast majority of cells. This can be done via an enrichment technique which uses a Ficoll Hypaque gradient centrifugation process to float the CTCs to the level of the buffy coat (FIG. 13), where cells with abundant cytoplasm, such as large mononuclear leukocytes, as well as CTCs band due to the effect of specific gravity (Katz et al., 2010; Katz and Lukeman, 1980). The buffy layers are aspirated with a Pasteur pipette and the total cell count can then be counted on Coulter instrument. Subsequently the concentration of cells is adjusted by using RPMI, or saline, so that the cells are well spread out with no overlapping, in order to allow the cells to be amenable to FISH studies or detection of membrane or cytoplasmic proteins such as different molecular weight of cytokeratins by immunocytochemistry.

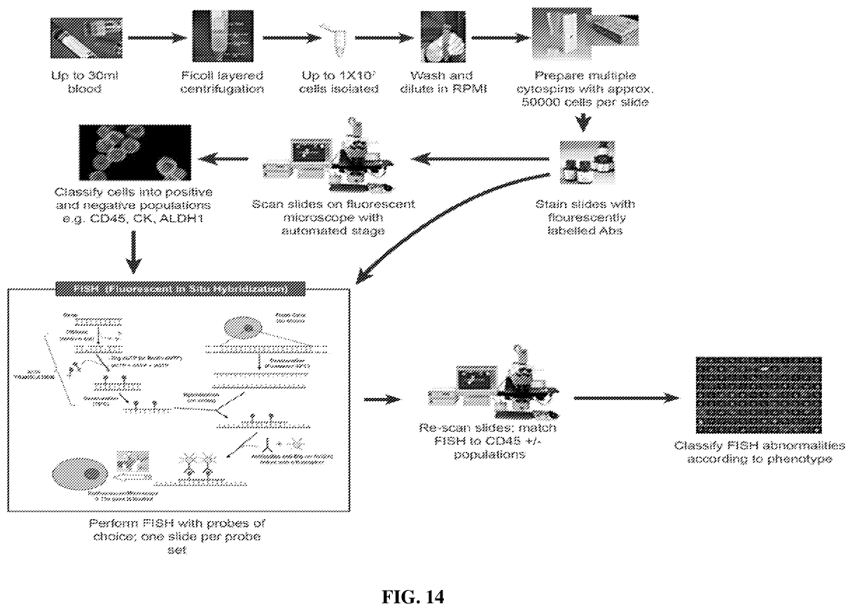

These preparations are then scanned automatically on an instrument composed of a fluorescent microscope with multiple filter wheels of different wavelengths to detect different color signals on an automated stage. An example of such an instrument is the Bioview Duet Instrument (Rehovoth, Il). This instrument can be programmed to select cells of a certain size and shape and exclude cells the size of a lymphocyte or smaller, so in effect a software filter is also present (WO2015103039). The instrument is capable of scanning and discerning the number of fluorescent signals per nucleus with great sensitivity and specificity, and at the end of each scan, produces a pie chart according to a classification such as: Single gain, (gain of a single signal), CTC class, defined as a gain of 2 or >signals from a minimum of 2 different probes counted per nucleus (FIG. 13), deletion, loss of a single signal. Using a four-color FISH probe, a diploid cell would therefore be defined as the presence of 8 signals, 2 red, 2 gold, 2 aqua and 2 green and a CTC would have at least 10 or more signals per nucleus, representing an extra gain from each DNA probe. However, the final reading and acceptance of the resultant FISH signal pattern is performed by a skilled and experienced operator, usually a cytotechnologist or cytogeneticist who will confirm that this is a true gain of signals and not due to overlapping cells. Such an assessment is best performed on the concurrent DAPI channel. This FISH based CTC method is possible to be performed fairly rapidly as cytospins can be loaded on an automatic stage and several thousand cells scanned within 15 minutes (FIGS. 13 and 14) (Jiang and Katz, 2002). Cells are only analyzed if they have intact nuclei that demonstrate good hybridization signals of all 4 probes tested.

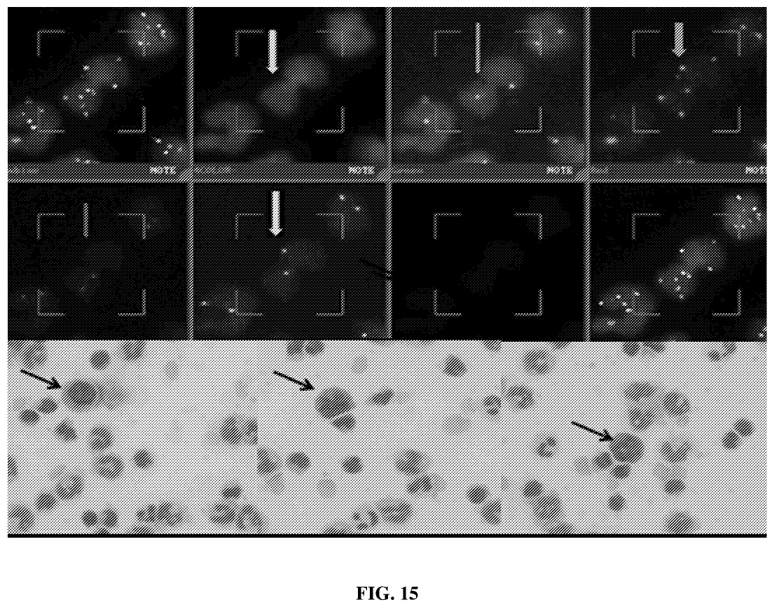

On the air-dried Diff-Quick stain, the cell morphology may vary from an oval or polygonal cell to, less frequently, cells that are spindle shaped and are consistent with cells that have undergone epithelial-to-mesenchymal transition. Cytoplasm may be more easily visualized compared to the neighboring hematopoietic-lymphoid component, with well-defined membranes and pale to basophilic homogeneous cytoplasm (FIG. 15). There may be a peri-nuclear clearing or “hof”. Nuclei may show patchy chromatin, with or without nucleoli, and be single, round or irregular, or occasionally binucleated, especially after chemotherapy that causes disruption of the microtubules in the spindle apparatus of the cell, preventing cytokinesis from taking place (FIG. 15B). Such cancer therapies include taxanes and nab-paclitaxel, docetaxel, vinblastine, vincristine and vinorelbine (Dean et al., 2001). The first 500 intact cells are analyzed by an operator and confirmed according to the classification of cells, beginning with the CTC class, followed by single gain and single deletion class, and then the class categorized “cell groups” and finally the “unclassified” class may be examined

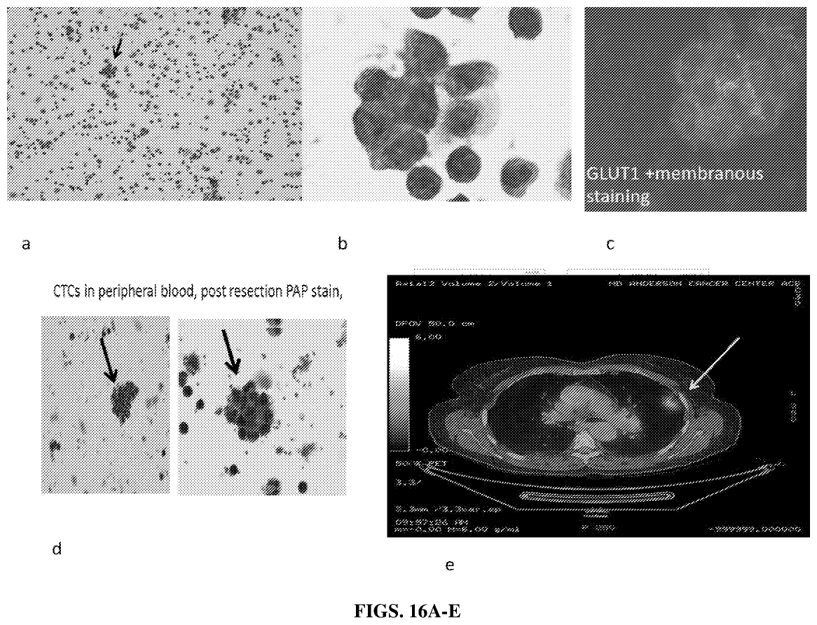

The goal of the analysis is to find as many unequivocal CTCs as possible as defined by polysomy of 2 or >signals of different nucleic acid probes per nucleus. A threshold for calling a specimen positive or negative is established based on the lowest number of aneuploid CTCs present in pathology proven cancer patients versus the highest number of aneuploid cells present in controls. In one study in patients with lung cancer, both small cell and non-small cell, the threshold chosen was ≥2 CTCs. which lead to a sensitivity of 88% and a specificity of 100% for the diagnosis of biopsy proven NSCLC. The optimal threshold is the one that most accurately predicts the presence of cancer. The goal is to produce as few false negative results or the highest negative predictive value as possible. It is also possible to combine the FISH genomic studies with prior or simultaneous immunohistochemistry for different cell surface markers such as cytokeratin, or CD45 or a metabolic marker such as GLUT1 (FIG. 16), a method known as FICTION (FIG. 14). See Ye X, Yan g Z, Carbone R et al. DOI: 10.5772/Intechopen.97631.

Validation studies proving the accuracy of the method have been performed by spiking H1299 lung adenocarcinoma cells into PBMNCs isolated from normal donors at different concentrations and hybridizing with a battery of DNA probes known to be abnormally expressed in the epithelial cells of lung cancer patients.⁹ The percentage of tumor cells recovered , that is cells showing aneuploidy by FISH, versus the percentage of tumor cells spiked in, is the recovery rate which can be plotted at different concentrations of spiked in H1299 cells.

FISH methods (WO2015103039 A1, incorporated herein by reference). The methods described by Katz et al. (WO2015103039) is fairly easy to perform, it can be scaled up with enhanced software and multiple reading stations connected to the main scanning instrument, and rapid FISH methodology takes only several hours of hybridization, instead of the traditional overnight hybridization can be used. Additionally, multiple unstained cytospins can be archived and used for other combinations of nucleic acid biomarkers, and/or combined with immunohistochemistry stains or rehybridized with the same DNA probes for interlab or intra-lab comparisons or quality improvement. This method has been validated recently by another outside lab on a split blood specimen. (Cynvenio, Inc., CA; unpublished data). A potential disadvantage of this method is that it requires a fresh blood specimen because optimal recovery the Ficoll procedure should be performed within 6 hours, but not more than 48 hours after collection.

Filtration Methods. CTCs are isolated by the size of epithelial cells (ISET) (Rarecells Diagnostics, Paris, France) via a blood filtration approach which enriches 10 ml of peripheral blood collected in buffered EDTA and kept at room temperature. Blood should be processed within one hour of collection. The membrane used is made of polycarbonate and allows cells<8 microns to pass through, while the larger epithelial and mononuclear white blood cells remain on top (FIG. 2C). The membrane can be cut in half, and half used for morphology via a May Grunewald Giemsa stain, and the second half can be used for immunocytochemistry using a pan-cytokeratin antibody and an anti-vimentin antibody applied to the filters. Malignant cells are identified according to usual characteristic features such as cell size, shape, nuclear cytoplasmic ratio, presence of nucleoli, cytoplasm, and expression of cytokeratin or vimentin or both antigens being co-expressed. Another similar platform is the CellSieve method that uses microfilters and a pressure monitored filtration pump (Creatv MicroTech, Inc, Rockville, Md.) (FIG. 10).

Single-Cell Genomic Analyses of CTCs. The rapid development of targeted therapies requires simultaneous detection of multiple molecular alterations. This poses a challenge for cytology, which traditionally examined cellular morphology and limited markers under the microscope. Recent progress in single-cell sequencing (SCS) could address the unmet need for highly multiplex testing in cytology. SCS provides genomic, transcriptomic, and methylomic information based on genetic material in a single cell. One key process in single-cell genome sequencing (SCGS) is to uniformly amply as little as 6 picogram of DNA to adequate quantity that is suitable for next-generation sequencing technologies. A few whole-genome amplification (WGA) methods have been developed. Among them, Φ29 polymerase-based isothermal amplification method, multiple displacement amplification (MDA) (Dean et al., 2001), offers low amplification uniformity and high coverage of the genome with low false positive and false negative rates in somatic mutation calling. The degenerate oligonucleotide-primed PCR (DOP-PCR) employed partially degenerated oligonucleotides in a PCR reaction to achieve high amplification uniformity and uniform genome coverage (Telenius et al., 1992). Quasi linear whole amplification methods, such as multiple annealing and looping-based amplification cycles (MALBAC), combined discrete steps of isothermal amplification with PCR reaction to yield uniform genome coverage with relatively low false positives and false negatives (Zong et al., 2012). A linear whole-genome amplification method, Linear

Amplification via Transposon Insertion (LIANTI), amplified the genomic DNA through in vitro transcription followed by reverse transcription (Chen et al., 2017). LIANTI, though tedious, exhibited so far the highest amplification uniformity and genome coverage, and lowest false positive rate.

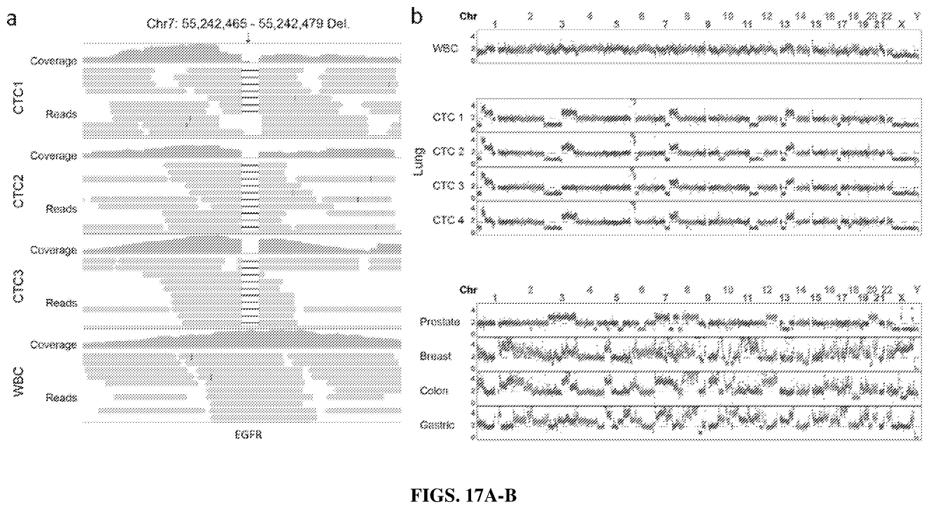

Capitalizing WGA's ability to achieve uniform genome coverage with high fidelity, SCGS was able to provide both somatic mutational profile and chromosomal aberrations profile in individual cells. As illustrated in FIG. 17⁵², whole-exome sequencing revealed that a 15-bp deletion in exon 19 of EGFR gene presented in three CTCs but not the white blood cell from a lung cancer patient (Ni et al., 2013). Whole-genome sequencing with sparse sequence coverage showed consistent copy number alterations (CNAs) in CTCs from a lung cancer patient and significant amplification in androgen receptor (AR) gene region of a CTC from a prostate cancer patient (Ni et al., 2013; Gao et al., 2017). Single-cell cancer genomics, since its debut in 2012, provided a deep understanding of cell-to-cell heterogeneity, cancer evolution, and metastatic process (Ni et al., 2013; Gao et al., 2017; Navin et al., 2011; Lowes et al., 2016; Hou et al., 2012; Francis et al., 2014; Leung et al., 2017).

Single-cell genomic analyses of CTCs have great potential to play an essential role in blood cytology for cancer patients. Therapeutically targetable genomic alterations could be monitored through repeated liquid biopsies by CTC sequencing (Ni et al., 2013; Heitzer et al., 2013; Lohr et al., 2014; Dago et al., 2014; Hodgkinson et al., 2014). CTC CNA analyses open a new avenue for molecular-based patient classification. In lung cancer, patients with small cell lung cancer were distinguished from patients with lung adenocarcinoma and a phenotypic transition from small cell lung cancer to lung adenocarcinoma in the same patient was informed based on CTC CNA profile (Ni et al., 2013). A CTC CNA-based classifier successfully identified 83.3% of SCLC patients as chemo refractory or chemosensitive (Carter et al., 2017).

Specific Organ Sites. Lung. The extremely high incidence of lung cancer world-wide, with its attendant high morbidity and mortality and frequent advanced stage presentation, when treatment and cure are usually not possible, presents an ideal scenario for instituting an accurate screening test for early lung cancer in a high-risk population. The National Lung Screening Trial (NLST), which enrolled 53,454 persons at high risk for lung cancer, was a randomized trial comparing low dose spiral CT scan with single view postero-anterior chest radiography over a 3 year period, and demonstrated that the mortality from lung cancer was reduced by 20% with the use of low-dose CT. Unfortunately low dose CT screening was associated with a high percentage of false positive result caused by benign etiologies such as resolving pneumonias or intra-pulmonary lymph nodes, or non-calcified granulomas (Aberle et al., 2011). A liquid biopsy approach using the presence of CTCs over a certain threshold to diagnose lung cancer as an adjunctive test in the presence of an indeterminate lung nodule, would be an ideal management tool for triage, if a sufficiently accurate assay could be discovered.⁹ Thus if the CTC test is positive, then a PET-CT scan followed by an FNA or thin-needle biopsy to obtain a tissue diagnosis would be desirable. If positive, then minimally invasive treatment options such as video-assisted thoroscopy, radio-frequency ablation or stereotaxic radiation therapy (SBRT) to deliver a focused intense course of XRT to obliterate the nodule would be advantageous.

CTCs may be detected in the peripheral blood of patients with advanced stage lung tumors but are usually not easily detected by in early lung cancer (Katz et al., 2010). The current FDA approved assay that relies on demonstration of EpCAM, is not approved for lung cancer, as metastatic lung cancer cells undergo epithelial mesenchymal transition, and may not express EpCAM. Patients at risk for lung cancer may present with indeterminate pulmonary nodules discovered during CT scan. However due to cost, morbidity, and high rate of negative biopsies, many patients are followed by CT scans alone. An accurate adjunctive biomarker that could predict for lung cancer in high-risk individuals with suspicious lung nodules would be advantageous for early diagnosis of lung cancer and for triaging these patients for biopsy. A prospective study using a custom made 4-color FISH probe, which was developed based on data derived from CGH arrays in Non-Small Cell Lung cancers (NSCLC) and which was designed to detect CTCs in the peripheral blood of NSCLC patients [ref 30] was carried out on patients with no prior history of LC, but who had an indeterminate lung nodule(s). Prior to needle biopsy of the nodule, 10 ml of blood was collected (Jiang et al., 2004). I-FISH was performed on blinded samples enriched for peripheral blood mononuclear cells using a custom probe set comprising 3q29 tel, 3p22.1, cep 10, and 10q22.3, the gene encoding the protein surfactant A. Intact cells (500) were analyzed by an automated instrument (Bioview Duet, Il) optimized to select for larger cells, and classified into subclasses based on gains and /or losses of fluorescent signals. CTCs were defined as cells with increased copy number of ≥2 genes. A positive assay was defined as ≥2 CTCs; negative assay was CTCs<2. The biopsy was used as the gold standard (FIG. 12). There were 30 true positive CTC assays that on biopsy were cases of NSCLC, and 6 false negative CTC assays in NSCLC patient's (2 neuroendocrine carcinomas, 3 adenocarcinomas, one squamous cell carcinoma). The sensitivity of the CTC test for diagnosing NSCLC was 88%, the specificity was 100%, the positive predictive value was 100%, and the negative predictive value was 88%. This is the first diagnostic blood biomarker to accurately detect early non-small cell lung cancer, as an adjunct to an indeterminate lung nodule, and hence has tremendous clinical utility. If validated extensively, this CTC test may substitute for a conventional biopsy, and may be used to guide therapy. While unable to detect 2 primary lung neuroendocrine neoplasms by the CTC test, very high numbers of single gains and losses of the FISH signals in many cells were noted. It was recommended that patients with a positive CTC test and an indeterminate lung nodule should be sent for fine needle aspiration and/or biopsy. Patients with a negative CTC tests may be able to avoid a potentially harmful biopsy and be followed clinically and by imaging studies.

In a side-by-side comparison of 40 patients with advanced NSCLC, predominantly stage IV, using the filtration-based size exclusion technology ISET (Rare Cell Diagnostics), investigators detected higher numbers of CTCs including epithelial marker-negative cells in 32/40 or 80% of patients as compared to CellSearch where only 9/40 or 23% of patients. were found to have CTCs In addition circulating tumor microemboli(CTM) were detected by filtration but not detectable by CellSearch (Katz et al., 2010; Krebs et al., 2014; Krebs et al., 2012). The blood specimens for each method comprised 10 ml of blood collected in CellSave tubes (Veridex, N.J.) for CellSearch and 10 ml in EDTA tubes for ISET. Contaminating skin cells, which may lead to false positive reports were rarely found.

Immunohistochemistry stains on cells isolated by ISET showed variable expression of EGFR, CK and Ki67; however, EpCAM was expression was not detectable (Krebs et al., 2012).

In a large study of patients with and without COPD, investigators noted that 5/168 patients with COPD or 3%, all of whom had negative spiral CT scans, manifested CTCs one to 4 years before the appearance of indeterminate lung nodules. All nodules proved to be early stage lung cancer on surgical resection (Ilie et al., 2014). The CTCs were isolated by the ISET filtration method and were stained with both epithelial and mesenchymal markers.

Expression of PDL-1 on NSCLC cells and the relationship between PDL-1 expression and its tumor environment characterized by tumor infiltrating lymphocytes, amongst other elements of the microenvironment, is a topic of immense interest in the backdrop of the stunning success that PDL1/PD1 immune checkpoint inhibitors (ICI) have had (such as pembrolizumab and nivolumab). According to PD-L1 status and number of TILs, a classification of tumors into four categories has been recently proposed (Dago et al., 2014; Hodgkinson et al., 2014). (This includes type I adaptive immune resistance (PD-L1-positive and high TILs), type II immune ignorance (PD-L1-negative and low TILs), type III intrinsic induction (PD-L1-positive and low TILs), and type IV immune tolerance (PD-L1-negative and high TILs (Mazzsachi et al., 2017). In the POPLAR trail, PD-L1 expression on NSCLC tumor tissue is predictive for benefit to PD-L1 inhibitor atezolizumab (Fehrenbacher et al., 2016). The PD-L1 status could be assessed from CTCs as the subtype of circulating lymphoid cells. One recent study from 106 NSCLC patients showed a 93% concordance between PD-L1 status in CTCs and tumor tissue, indicating the potential of CTC test in determining response to ICI (Ilie et al., 2018). Currently, CTC PD-L1 test is commercially available (such as Cynvenio and Biocept).

Breast. By far the most established clinical application for CTCs is its application in breast cancer with the recognition that in stage IV breast cancer, the number of CTCs enumerated by the Cell Search platform if ≥5, is significantly associated with a poorer prognosis. In the SUCCESS (Simultaneous Study of Gemcitabine-Docetaxel Combination adjuvant treatment, as well as Extended Bisphosphonate and Surveillance-Trial) trial (Rack et al., 2014), CTCs as measured by the CellSearch system were statistically significantly associated with node-positive disease. The presence of CTCs both before the start of systemic adjuvant treatment and after completion of chemotherapy was associated with deteriorated survival. Prognostic relevance independent of lymph node metastases was confirmed in multivariate analysis.

Both trials demonstrated the prognostic relevance of CTCs in early breast cancer despite low numbers of cells detected by the CellSearch system that were limited to cells with expression of Epcam and cytokeratin. However basal-like (triple-negative) tumors with low Epcam expression have been shown to contain a high frequency of stem cells and are associated with very poor prognosis CTCs with decreased epithelial marker expression as a result of the epithelial-mesenchymal transition could be missed by the CellSearch methodology. Epcam-independent detection approaches could increase the capacity to detect CTCs with a stem cell phenotype (Rack et al., 2014).

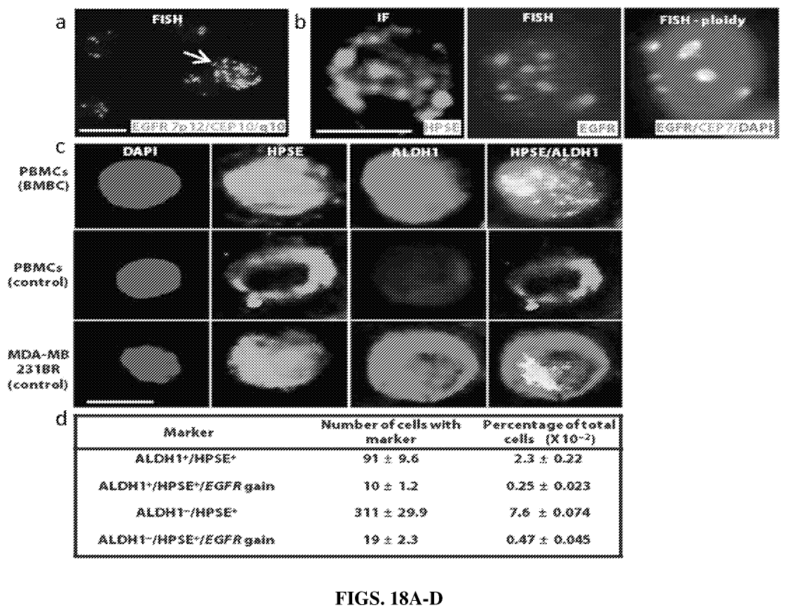

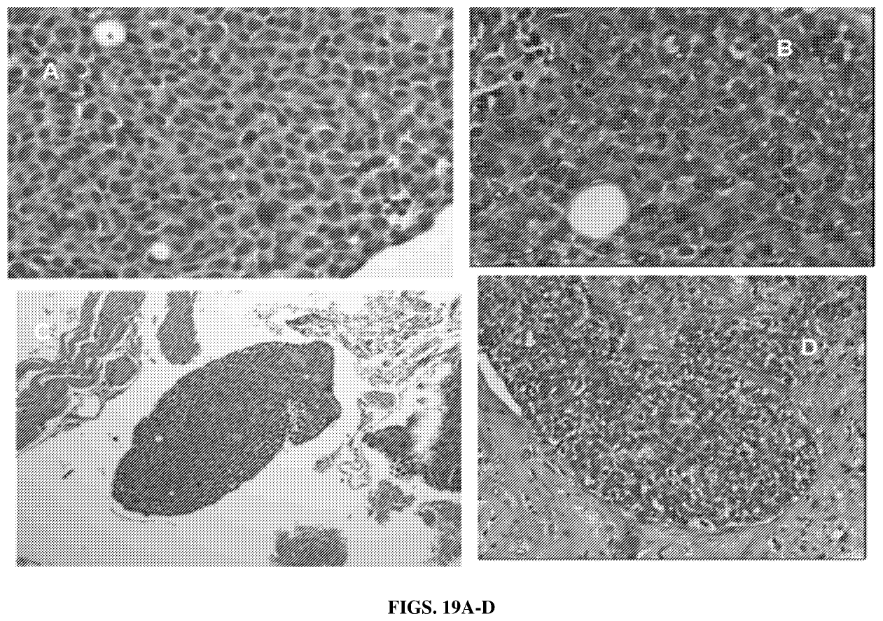

In an effort to look for the genomic signatures of breast cancer cells that metastasize to brain (BCBM), a very poor prognostic category, investigators employed an EpCAM independent approach which the inventors developed in their laboratory (Kim et al., 2009). The inventors used immunofluorescence for cytokeratin, combined with a custom probe for CEP 10, 10q22.3 and EGFR (Cytocell) and originally synthesized for the inventors' lung cancer studies, to analyze blood from patients with breast cancer metastatic to the brain (BCMB). They showed that CTCs in these patients were EpCAM-negative, EGFR amplified, expressed heparinase (HPSE), and ALDH1, a stem cell marker (FIG. 18). Concurrent blood samples run on the CellSearch system showed that the IFL (FICTION) assay recovered many more CTCs compared to the CellSearch platform which relied on EpCAM positivity (FIG. 18) (Zhang et al., 2013). They showed that CTCs from BCMB were EpCAM-negative, EGFR-amplified, expressed HPSE, and ALDH1. Using FACs analysis sorted cells that were ALDH1+, CD45− and EpCAM from patients' blood showed that patients who have BCMB have a specific signature that enables such CTCs to develop competency to metastasize to brain. Parental CTCs that were EGFR+/ALDH1+/CD45−/EpCAM from patients with BCBM were cultured. Clones were selected for expression of different biomarkers: HER2+/EGFR+/HPSE+/Notch1 and injected into mice, 60%-80% of these cells metastasized to brains of mice compared to only 0-20% for parental lines (FIGS. 19A-D).

Conclusion. CTCs appear to have tremendous potential for clinical usage and for detecting, monitoring and treating a variety of different cancers. The rate limiting factor in many studies has been the scarcity of recovery of CTCs and their downstream molecular analysis, as well as many different methods for detecting and capturing CTCs and lack of standardization and clinical validation. However the field is rapidly evolving and new discoveries and applications are being frequently reported, most importantly being the ability to dissect the genome of CTCs in “real time” by whole genome amplification on a single cell basis, providing unprecedented opportunities for targeted therapy as well as new antigen-independent methods for discovering CTCs in early lung cancer. Following extensive clinical validation, the inventors believe that there is great potential for incorporating these novel and minimally invasive tests into daily practice in the fields of cancer prevention, detection and monitoring response to therapy.

Table 1. Normal, diploid cells or eight signals according to interphase FISH. Deletion, single, comprising loss of signal compared with the centromeric probe; Gain, comprising an extra signal compared with the centromeric probe; CTC, comprising at least two gains of signals per nucleus (total, >10 signals).

Table 2. Frequency of genetic abnormalities identified using four-color FISH in patients without lung cancer (controls), with lung cancer (cases), by stage of lung cancer (stage 1-IV), and in the discovery and validation cohorts. *P<0.05. **P<0.01. ***P<0.001. Values are shown as percentage and number of CTC and all other cellular genetic abnormalities/500 cells counted and expressed as IQR (interquartile range).

TABLE 1
Results of spiking different concentrations of A549
LC cells into PBMC using a four-color FISH probe

| | | | | | Expected | Actual | | Number |
|---|---|---|---|---|---|---|---|---|
| Experimental | | | | CTC | tumor cell | tumor cell | | of cells |
| System | Normal | Deletion | Gain | (%) | recovery | recovery | Yield | analyzed |
| Unspiked | 99.6 | 0.22 | 0.22 | 0 | 0 | 0.44 | 0 | 500 |
| PBMC | | | | | | | | |
| A549 | 0.2 | 0 | 0 | 99.8 | 100 | 99.80 | 99.8 | 500 |
| cells | | | | | | | | |
| Four-color | 98.3 | 0.60 | 0 | 0.8 | 1 | 0.80 | 80.0 | 500 |
| probe, | | | | | | | | |
| 1% dilution | | | | | | | | |
| Four-color | 88.8 | 0.40 | 4.20 | 6.6 | 10 | 6.60 | 66.0 | 500 |
| probe, | | | | | | | | |
| 10% dilution | | | | | | | | |

TABLE 2
Prevalence of circulating tumor cells and all other cellular genetic abnormalities

| | Controls | Cases | Stage I | Stage II | Stage III | Stage IV | Discovery | Validation |
|---|---|---|---|---|---|---|---|---|
| Cellular characteristics | (N = 107) | (N = 100) | (N = 55) | (N = 10) | (N = 18) | (N = 17) | (N = 118) | (N = 89) |
| 3q29 | | | | | | | | |
| 3q29 deletion (%), | 0.22 | 0.26 | 0.21 | 0.2 | 0.61 | 0.2 | 0.2 | 0.45 |
| Median (IQR) | (0-0.64) | (0-0.62) | (0-0.6) | (0-0.62) | (0.2-0.87) | (0-0.6) | (0-0.41) | (0.2-0.81)*** |
| 3q29 deletion (N), | 1 | 1 | 1 | 1 | 3 | 1 | 1 | 2 |
| Median (IQR) | (0-3) | (0-3) | (0-3) | (0-3) | (1-4) | (0-3) | (0-2) | (1-4)*** |
| 3q29 gain (%), | 0.4 | 0.47 | 0.45 | 1.1 | 0.415 | 0.43 | 0.405 | 0.41 |
| Median (IQR) | (0-0.72) | (0.2-1.09)* | (0.2-1.08) | (0.2-1.6) | (0.2-0.8) | (0-1.2) | (0.12-1.08) | (0.1-0.8) |
| 3q29 gain (N, | 2 | 2 | 2 | 5.5 | 2 | 2 | 2 | 2 |
| Median (IQR) | (0-3) | (1-5)** | (1-5) | (1-8) | (1-4) | (0-6) | (1-5) | (1-4) |
| 3p22.1 | | | | | | | | |
| 3p22.1 deletion (%), | 0.2 | 0.21 | 0.2 | 0.205 | 0.1 | 0.25 | 0.2 | 0.22 |
| Median (IQR) | (0-0.4) | (0-0.6)* | (0-0.6) | (0.2-0.61) | (0-0.47) | (0-0.46) | (0-0.4) | (0-0.45) |
| 3p22.1 deletion (N), | 1 | 1 | 1 | 1 | 0.5 | 1 | 1 | 1 |
| Median (IQR) | (0-2) | (0-3)* | (0-3) | (1-3) | (0-2) | (0-2) | (0-2) | (0-2) |
| 3p22.1 gain (%), | 0.2 | 0.2 | 0.2 | 0.305 | 0.2 | 0.23 | 0.1 | 0.21 |
| Median (IQR) | (0-0.4) | (0-0.4) | (0-0.4) | (0-0.6) | (0-0.22) | (0-0.4) | (0-0.4) | (0-0.4)* |
| 3p22.1 gain (N), | 1 | 1 | 1 | 1.5 | 1 | 1 | 0.5 | 1 |
| Median (IQR) | (0-2) | (0-2) | (0-2) | (0-3) | (0-1) | (0-2) | (0-2) | (0-2) |
| CEP10 | | | | | | | | |
| CEP10 deletion (%), | 0.455 | 0.84 | 1 | 0.2 | 0.84 | 1 | 0.65 | 0.62 |
| Median (IQR) | (0.21-1) | (0.21-1.65)** | (0.4-1.8) | (0-0.83) | (0.2-1.05) | (0.4-1.8) | (0.2-1.4) | (0.23-1.07) |
| CEP10 deletion (N), | 2 | 4 | 5 | 1 | 4 | 5 | 3 | 3 |
| Median (IQR) | (1-4.5) | (1-8)** | (2-9) | (0-4) | (1-5) | (2-9) | (1-6) | (1-5) |
| CEP10 gain (%), | 0.24 | 0.4 | 0.4 | 0.4 | 0.415 | 0.23 | 0.2 | 0.42 |
| Median (IQR) | (0-0.6) | (0.2-0.62) | (0-0.6) | (0-0.41) | (0.2-1) | (0-0.42) | (0-0.42) | (0.22-0.68)*** |
| CEP10 gain (N), | 1 | 2 | 2 | 2 | 2 | 1 | 1 | 2 |
| Median (IQR) | (0-3) | (1-3) | (0-3) | (0-2) | (1-5) | (0-2) | (0-2) | (1-3)*** |
| 10q22.3 | | | | | | | | |
| 10q22.3 deletion (%), | 0 | 0.2 | 0.1 | 0.205 | 0.2 | 0.2 | 0 | 0 |
| Median (IQR) | (0-0.2) | (0-0.24)*** | (0-0.4) | (0.2-0.6) | (0-0.21) | (0-0.24) | (0-0.21) | (0-0.21) |
| 10q22.3 deletion (N), | 0 | 1 | 1 | 1 | 1 | 1 | 0 | 0 |
| Median (IQR) | (0-1) | (0-1)*** | (0-1) | (1-3) | (0-1) | (0-2) | (0-1) | (0-1) |
| 10q22.3 gain (%), | 0.2 | 0.41 | 0.4 | 0.41 | 0.525 | 0.6 | 0.22 | 0.22 |
| Median (IQR) | (0-0.425) | (0.2-0.8)*** | (0-0.64) | (0.2-1) | (0.2-0.84) | (0.4-0.8) | (0-0.64) | (0-0.6) |
| 10q22.3 gain (N), | 1 | 2 | 1 | 2 | 2.5 | 3 | 1 | 1 |
| Median (IQR) | (0-2) | (1-4)*** | (0-3) | (1-5) | (1-4) | (2-4) | (0-3) | (0-3) |
| CTC (%), | 0 | 0.94 | 0.82 | 1.4 | 0.905 | 0.95 | 0.53 | 0.4 |
| Median (IQR) | (0-0.21) | (0.63-1.2)*** | (0.61-1.06) | (1.03-1.6) | (0.8-1.2) | (0.6-1.07) | (0-1.04) | (0.2-0.8) |
| CTC (N), | 0 | 4 | 4 | 7 | 4 | 5 | 2 | 2 |
| Median (IQR) | (0-1) | (3-5)*** | (3-5) | (4-8) | (4-6) | (4-5) | (0-5) | (1-4) |
| Total | 13 | 23 | 22 | 28.5 | 21.5 | 24 | 17 | 18 |
| abnormalities (N), | (10-16) | (19-29)*** | (18-27) | (26-30) | (19-27) | (20-29) | (11-25) | (14-22) |
| Median (IQR) | | | | | | | | |
| Total | 2.725 | 4.75 | 4.5 | 5.8 | 4.43 | 5.07 | 3.665 | 3.74 |
| abnormalities (%), | (2.17-3.445) | (3.84-5.88)*** | (3.67-5.6) | (5.2-6.6) | (3.8-5.65) | (4-5.8) | (2.4-5.4) | (2.9-4.6) |
| Median (IQR) | | | | | | | | |

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
1. Pantel K, Brakenhoff R H. Dissecting the metastatic cascade. Nature reviews Cancer 2004; 4:448-56.
2. Husemann Y, Geigl J B, Schubert F, et al. Systemic spread is an early step in breast cancer. Cancer cell 2008; 13:58-68.
3. Rhim A D, Mirek E T, Aiello N M, et al. EMT and dissemination precede pancreatic tumor formation. Cell 2012; 148:349-61.
4. Sanger N, Effenberger K E, Riethdorf S, et al. Disseminated tumor cells in the bone marrow of patients with ductal carcinoma in situ. International journal of cancer 2011; 129:2522-6.
5. He W, Wang H, Hartmann L C, Cheng J X, Low P S. In vivo quantitation of rare circulating tumor cells by multiphoton intravital flow cytometry. Proceedings of the National Academy of Sciences of the United States of America 2007; 104:11760-5.
6. Pantel K, Speicher M R. The biology of circulating tumor cells. Oncogene 2016; 35:1216-24.
7. Hanahan D, Weinberg R A. Hallmarks of cancer: the next generation. Cell 2011; 144:646-74.
7a. Helei Hou, Chuantao Zhang, Dong Lui, Na Zhou, Xiaochun Zhang, Discovery of targetable genetic alterations in advanced non-small cell lung cancer using a next-generation circulating tumor DNA assay. Scientific Reports, 7, article number: 14605 (2017)]
8. Ni X, Zhuo M, Su Z, et al. Reproducible copy number variation patterns among single circulating tumor cells of lung cancer patients. Proceedings of the National Academy of Sciences of the United States of America 2013; 110:21083-8.
9. Katz R L, He W, Khanna A, et al. Genetically abnormal circulating cells in lung cancer patients: an antigen-independent fluorescence in situ hybridization-based case-control study. Clinical cancer research : an official journal of the American Association for Cancer Research 2010; 16:3976-87.
10. Krebs M G, Metcalf R L, Carter L, Brady G, Blackhall F H, Dive C. Molecular analysis of circulating tumour cells-biology and biomarkers. Nature reviews Clinical oncology 2014; 11:129-44.
10a. Krebs, Matthew G, Hou Jian-Mei, Sloane Robert et al. Analysis of Circulating Tumor cells in Patients with Non-small Cell Lung Cancer Using Epithelial Marker-Dependent and -Independent Approaches, J Thorac Oncol 2012; 7;306-315).
11. Hou J M, Krebs M G, Lancashire L, et al. Clinical significance and molecular characteristics of circulating tumor cells and circulating tumor microemboli in patients with small-cell lung cancer. Journal of clinical oncology : official journal of the American Society of Clinical Oncology 2012; 30:525-32.
12. Meng S, Tripathy D, Frenkel E P, et al. Circulating tumor cells in patients with breast cancer dormancy. Clinical cancer research: an official journal of the American Association for Cancer Research 2004; 10:8152-62.
13. Kim M Y, Oskarsson T, Acharyya S, et al. Tumor self-seeding by circulating cancer cells. Cell 2009; 139:1315-26.
14. Zhang L, Ridgway L D, Wetzel M D, et al. The identification and characterization of breast cancer CTCs competent for brain metastasis. Science translational medicine 2013; 5:180ra48.
15. Maheswaran S, Haber D A. Circulating tumor cells: a window into cancer biology and metastasis. Current opinion in genetics & development 2010; 20:96-9.
16. Tang C M, Zhu P, Li S, Makarova O V, Amstutz P T, Adams D L. Filtration and Analysis of Circulating Cancer Associated Cells from the Blood of Cancer Patients. Methods in molecular biology (Clifton, N.J.) 2017; 1572:511-24.
17. Kinio S, Mills J K. Localized Electroporation with Dielectrophoretic Field Flow Fractionation: Towards Removal of Circulating Tumour Cells from Human Blood. IEEE transactions on nanobioscience 2017: 1.
18a Miller M C, Doyle G V, Terstappen L W. Significance of Circulating Tumor Cells Detected by the CellSearch System in Patients with Metastatic Breast Colorectal and Prostate Cancer. Journal of oncology 2010; 2010:617421.
18b. Pachmann U A, Hekimian K, Carl S, Ruediger N, Rabenstein C, Pachmann K, Comparing Sequential Steps for Detection of Circulating Tumor Cells: More specific or just less sensitive? Cancer 2011; 2(2) WMC001490.
19. Riethdorf S, Muller V, Zhang L, et al. Detection and HER2 expression of circulating tumor cells: prospective monitoring in breast cancer patients treated in the neoadjuvant GeparQuattro trial. Clinical cancer research: an official journal of the American Association for Cancer Research 2010; 16:2634-45.
20. de Bono J S, Attard G, Adjei A, et al. Potential applications for circulating tumor cells expressing the insulin-like growth factor-I receptor. Clinical cancer research: an official journal of the American Association for Cancer Research 2007; 13:3611-6.
21. Mikolajczyk S D, Millar L S, Tsinberg P, et al. Detection of EpCAM-Negative and Cytokeratin-Negative Circulating Tumor Cells in Peripheral Blood. Journal of oncology 2011; 2011:252361.
22. Grover P K, Cummins A G, Price T J, Roberts-Thomson I C, Hardingham J E. Circulating tumour cells: the evolving concept and the inadequacy of their enrichment by EpCAM-based methodology for basic and clinical cancer research. Annals of oncology: official journal of the European Society for Medical Oncology 2014; 25:1506-16.
23. Gabriel M T, Calleja L R, Chalopin A, Ory B, Heymann D. Circulating Tumor Cells: A Review of Non-EpCAM-Based Approaches for Cell Enrichment and Isolation. Clinical chemistry 2016; 62:571-81.
24. Liu Z, Fusi A, Klopocki E, et al. Negative enrichment by immunomagnetic nanobeads for unbiased characterization of circulating tumor cells from peripheral blood of cancer patients. Journal of translational medicine 2011; 9:70.
25. Lapin M, Tjensvoll K, Oltedal S, et al. MINDEC-An Enhanced Negative Depletion Strategy for Circulating Tumour Cell Enrichment. Scientific reports 2016; 6:28929.
26. Wu Y, Schoenborn J R, Morrissey C, et al. High-Resolution Genomic Profiling of Disseminated Tumor Cells in Prostate Cancer. The Journal of molecular diagnostics: JMD 2016; 18:131-43.
27. Scher H I, Graf R P, Schreiber N A, et al. Phenotypic Heterogeneity of Circulating Tumor Cells Informs Clinical Decisions between AR Signaling Inhibitors and Taxanes in Metastatic Prostate Cancer. Cancer Res 2017; 77:5687-98.
28. Katz R L, Lukeman J M. The comparative diagnostic accuracy of cancer-cell detection obtained with Ficoll-Hypaque gradient separation and standard centrifugation technics on body-cavity fluids. Am J Clin Pathol 1980; 74:18-24.
29. Jiang F, Katz R L. Use of interphase fluorescence in situ hybridization as a powerful diagnostic tool in cytology. Diagnostic molecular pathology: the American journal of surgical pathology, part B 2002; 11:47-57.
30. Jiang F, Qiu Q, Khanna A, et al. Aldehyde dehydrogenase 1 is a tumor stem cell-associated marker in lung cancer. Mol Cancer Res 2009; 7:330-8.
31. Dean F B, Nelson J R, Giesler T L, Lasken R S. Rapid amplification of plasmid and phage DNA using Phi 29 DNA polymerase and multiply-primed rolling circle amplification. Genome Res 2001; 11:1095-9.
32. Telenius H, Carter N P, Bebb C E, Nordenskjold M, Ponder B A, Tunnacliffe A. Degenerate oligonucleotide-primed PCR: general amplification of target DNA by a single degenerate primer. Genomics 1992; 13:718-25.
33. Zong C, Lu S, Chapman A R, Xie X S. Genome-wide detection of single-nucleotide and copy-number variations of a single human cell. Science (New York, N.Y.) 2012; 338:1622-6.
34. Chen C, Xing D, Tan L, et al. Single-cell whole-genome analyses by Linear Amplification via Transposon Insertion (LIANTI). Science (New York, N.Y.) 2017; 356:189-94.
35. Gao Y, Ni X, Guo H, et al. Single-cell sequencing deciphers a convergent evolution of copy number alterations from primary to circulating tumor cells. Genome Res 2017; 27:1312-22.
35a. WO2015103039 A1
36. Navin N, Kendall J, Troge J, et al. Tumour evolution inferred by single-cell sequencing. Nature 2011; 472:90-4.
36a. Lowes L E, Bratman S V, Dittamore R, el al. Circulating Tumor Cells (CTC) and Cell-Free DNA (cfDNA) Workshop 2016: Scientific Opportunities and Logistics for Cancer Clinical Trial Incorporation. Int J Mol Sci. 2016; 17(9):1505. Published 2016 Sep. 8. doi:10.3390/ijms17091505.
37. Hou Y, Song L, Zhu P, et al. Single-cell exome sequencing and monoclonal evolution of a JAK2-negative myeloproliferative neoplasm. Cell 2012; 148:873-85.
38. Francis J M, Zhang C Z, Maire C L, et al. EGFR variant heterogeneity in glioblastoma resolved through single-nucleus sequencing. Cancer Discov 2014; 4:956-71.
39. Leung M L, Davis A, Gao R, et al. Single-cell DNA sequencing reveals a late-dissemination model in metastatic colorectal cancer. Genome Res 2017; 27:1287-99.
40. Heitzer E, Auer M, Gasch C, et al. Complex tumor genomes inferred from single circulating tumor cells by array-CGH and next-generation sequencing. Cancer Res 2013; 73:2965-75.
41. Lohr J G, Adalsteinsson V A, Cibulskis K, et al. Whole-exome sequencing of circulating tumor cells provides a window into metastatic prostate cancer. Nat Biotechnol 2014; 32:479-84.
42. Dago A E, Stepansky A, Carlsson A, et al. Rapid phenotypic and genomic change in response to therapeutic pressure in prostate cancer inferred by high content analysis of single circulating tumor cells. PloS one 2014; 9:e101777.
43. Hodgkinson C L, Morrow C J, Li Y, et al. Tumorigenicity and genetic profiling of circulating tumor cells in small-cell lung cancer. Nature medicine 2014; 20:897-903.
44. Carter L, Rothwell D G, Mesquita B, et al. Molecular analysis of circulating tumor cells identifies distinct copy-number profiles in patients with chemosensitive and chemorefractory small-cell lung cancer. Nature medicine 2017; 23:114-9.
45. Aberle D R, Adams A M, Berg C D, et al. Reduced lung-cancer mortality with low-dose computed tomographic screening. The New England journal of medicine 2011; 365:395-409.
46. Jiang F, Yin Z, Caraway N P, Li R, Katz R L. Genomic profiles in stage I primary non small cell lung cancer using comparative genomic hybridization analysis of cDNA microarrays. Neoplasia (New York, N.Y.) 2004; 6:623-35.
47. Ilie M, Hofman V, Long-Mira E, et al. “Sentinel” circulating tumor cells allow early diagnosis of lung cancer in patients with chronic obstructive pulmonary disease. PloS One 2014; 9:e111597.
48. Mazzaschi G, Madeddu D, Falco A, et al. Low PD-1 expression in Cytotoxic CD8+ Tumor infiltrating Lymphocytes Confers an Immune Privileged Tissue Microenvironment in NSCLC with a Prognostic and Predictive Value. Clinical cancer research: an official journal of the American Association for Cancer Research 2017.
49. Fehrenbacher D, et al., Lancet 2016; 387; 1837).
50. Ilie M, et al., Annals of Oncology, 2018, doi.org/10.1093/annonc/mdx636.
51. Rack B, Schindlbeck C, Juckstock J, et al. Circulating tumor cells predict survival in early average-to-high risk breast cancer patients. Journal of the National Cancer Institute 2014; 106.
52. Katz R L, Zaidi T M, Xiaohui N, Liquid Biopsy: Recent advances in the detection of circulating tumor cells and their clinical applications. Bui M M, Pantanowitz (eds): Modern Techniques in Cytopathology. Monogr Clin Cytol Basel, Karger, 2020, vol 25, pp 43-66 (DOI: 10.1159/000455780).
53. Xin Ye, Xiao Zheng Yang, Roberta Carbone, Iris Barshack and Ruth L. Katz (May 31, 2021). Diagnosis of Non-Small Cell Lung Cancer via Liquid Biopsy Highlighting a Fluorescence-in-situ-Hybridization Circulating Tumor Cell Approach [Online First], IntechOpen, DOI: 10.5772/intechopen.97631; world-wide-web at intechopen.com/online-first/diagnosis-of-non-small-cell-lung-cancer-via-liquid-biopsy-highlighting-a-fluorescence-in-situ-hybrid.

1. A method of detecting a circulating tumor cell (CTC) in a fluid sample comprising:
(a) obtaining a cell-containing fluid sample from a subject, wherein said fluid sample is buffy coat portion of a blood sample;
(b) performing an ambient ionization MS on the sample to obtain a profile for the sample; and
(c) detecting the presence of a CTC based on the profile.

2. The method of claim 1, wherein said sample is disposed on a surface and said method further comprises marking one or more regions on said surface corresponding to a detected CTC.

3. The method of claim 1, further comprising hybridizing the detected abnormal cells in said sample with labeled nucleic acid probes for (a) 3p22.1, 10q22.3, chromosome 10 centromeric (cep10) and (b) chromosome 3 centromeric (cep3) or 3q29 tel, and detecting CTCs based on pattern of hybridization to all four labeled nucleic acid probes to said detected abnormal cells.

4. The method of claim 3, wherein the label is a fluorescent label or a chromogenic label.

5. The method of claim 1, further defined as a method for detecting cancer in the subject.

6. The method of claim 1, wherein the CTCs are from a cancer that gives rise to blood borne metastases, such as cancer of lung, head and neck, thyroid, breast, colon, prostate, pancreas, esophagus, kidney, a gastro-intestinal tumor, a urogenital tumor, kidney, a melanoma, an endocrine tumor or a sarcoma or circulating malignant cells derived from a leukemia or a lymphoma.

7. The method of claim 1, further comprising detecting hybridization of one or more additional labeled nucleic acid probes, such as a UroVysion DNA probe set, a LaVysion DNA probe set, a centromeric 7/7p12 Epidermal Growth Factor (EGFR) probe, cep7/7p22.1, cep17, and 9p21.3 probes, EGFR/cep and 10/cep10q probes, pTEN, cep10 and cep10q probes, an EML4-ALK probe set, a cytoplasmic probe such as microRNA probe such as miRNA21, a surface or cytoplasmic biomarker probe such as ER,PR, Her2neu, a pan-cytokeratin, or a CA19.

8. The method of claim 1, wherein the ambient ionization MS comprises DESI-MSI.

9. The method of claim 8, comprising performing 2D DESI-MSI, such as wherein 2D DESI-MSI comprises a spatial resolution of 500 μm to 50 μm.

10. The method of claim 1, further comprising obtaining a reference profile and detecting the presence of CTCs by comparing the profile from the sample to a reference profile.

11. The method of claim 10, wherein the reference profile is obtained from the same subject.

12. The method of claim 10, wherein the reference profile is obtained from a different subject.

13. The method of claim 1, wherein the profile comprises fatty acid and metabolite molecules and species with m/z of 215.033, 255.233, 283.264, and 303.233.

14. The method of claim 1, wherein the profile comprises ceramide species with m/z ratios of 572.48, 656.578, and 682.594.

15. The method of claim 1, wherein the profile comprises glycerophosphoethanolamine molecules and species with m/z 722.513, 750.546, and 766.542.

16. The method of claim 1, wherein the profile comprises cardiolipin molecules and species with m/z 723.499, and 725.495.

17. The method of claim 1, wherein the profile comprises glycerophosphoserine molecules and species with m/z 788.545, and 810.529.

18. The method of claim 1, wherein the profile comprises glycerophosphoglycerol molecules and species with m/z 747.520, 773.534.

19. The method of claim 1, wherein the profile comprises glycerophosphoinositol molecules and species with m/z 835.535, 857.520, 861.551, and 885.550.

20. The method of claim 1, further comprising filtering said fluid sample.

21. The method of claim 20, wherein filtering comprises use of a vacuum apparatus and a membrane perforated with 7.5 μm pores.

22. The method of claim 1, wherein the buffy coat layer is separated from the blood by a Ficoll-Hypaque gradient.

23. The method of claim 1, wherein the buffy coat layer is further purified by CD45 bead-based purification to remove white blood cells.

24. The method of claim 1, wherein the buffy coat layer is further purified by CD3 bead-based purification to remove white blood cells.

25. The method of claim 1, wherein the buffy coat layer is separated from the blood by a Ficoll-Hypaque gradient, is further purified by CD45 bead-based purification to remove white blood cells and is further purified by CD3 bead-based purification to remove white blood cells.

26. The method of claim 1, further comprising collecting the sample from the subject.

27. The method of claim 1, further comprising selecting CTCs by assessing nuclear area comprises determining pixel size for each CTC and applying a pre-determined threshold for exclusion, and or determining nuclear diameter and/or determining DAPI concentration and its standard deviation.

28. The method of claim 1, wherein detecting comprises assessing all abnormalities or gains only.

29. The method of claim 1, further comprising:
(c) administering at least a first anticancer therapy to a subject identified to have CTCs.

30. The method of claim 29, wherein the anticancer therapy comprises radiation, immunotherapy, toxin therapy, hormonal therapy, surgery or chemotherapy therapy.

31. The method of claim 30, wherein the anticancer therapy is a combination therapy comprising more than one of radiation, immunotherapy, toxin therapy, hormonal therapy, surgery and chemotherapy therapy.

32. A method of treating a subject comprising:
(a) selecting a patient determined to have CTCs in accordance with claim 1; and
(b) administering at least a first anticancer therapy to the subject.

33. The method of claim 32, wherein the anticancer therapy comprises radiation, immunotherapy, surgery or chemotherapy therapy.

34. The method of claim 32, wherein the cancer is a one that gives rise to blood borne metastases, such as cancer of lung, head and neck, breast, colon, prostate, pancreas, esophagus, kidney, a gastro-intestinal tumor, a urogenital tumor, kidney, a melanomas, an endocrine tumor (thyroid, e.g., papillary thyroid cancer (PTC) adrenal gland cortex or medulla) or a sarcoma, or leukemia or lymphoma.
